(19) United States
(12) Patent Application Publication
(10) Pub. No.: US 2026/0240869 A1
(43) Pub. Date: Aug. 20, 2026

(54) ANTIPSEUDOMONAL FUSIDIC ACID COMPOUNDS

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Martin GARCIA CHAVEZ, Urbana, IL (US)

(73) Assignee: THE BOARD OF TRUSTEES OF THE UNIVERSITY OF ILLINOIS, Urbana, IL (US)

(21) Appl. No.: 18/876,672

(22) PCT Filed: Jun. 28, 2023
(86) PCT No.: PCT/US2023/069237

§ 371 Date: Dec. 19, 2024

Related U.S. Application Data
(60) Provisional application No. 63/356,198, filed on Jun. 28, 2022.

(51) Int. Cl. A61K 31/565; A61K 31/58; A61P 31/04; C07J 1/00; C07J 43/00

(52) U.S. Cl. CPC A61K 31/565; A61K 31/58; A61P 31/04; C07J 1/00; C07J 43/003

(57) ABSTRACT

The whole-cell accumulation of 345 diverse compounds in aeruginosa and E. coli have been assessed. While certain positively charged compounds were demonstrated to permeate both bacterial species, P. aeruginosa is more restrictive as compared to E. coli. Computational analysis identified distinct physicochemical properties of small molecules that are specifically correlated with P. aeruginosa accumulation, such as formal charge, positive polar surface area, and hydrogen bond donor surface area. Mode of uptake studies revealed that most small molecules permeate P. aeruginosa using a porin-independent pathway, thus enabling discovery of general P. aeruginosa accumulation trends with important implications for future antibiotic development. These discoveries were then applied to expand the spectrum of activity of a Gram-positive-only antibiotic, fusidic acid, into a version that demonstrates a 256-fold improvement in antibacterial activity against P. aeruginosa including dozens of clinical isolates.

### RELATED APPLICATIONS

This application claims priority under 35 U.S.C. § 119(e) to U.S. Provisional Patent Application No. 63/356,198, filed Jun. 28, 2022, which is incorporated herein by reference.

### GOVERNMENT SUPPORT

This invention was made with government support under R01AI136773 awarded by the National Institutes of Health. The government has certain rights in the invention.

### BACKGROUND OF THE INVENTION

The World Health Organization and Centers for Disease Control and Prevention have identified Gram-negative Enterobacteriaceae (E. coli, K. pneumoniae, etc.), Acinetobacter baumannii, and Pseudomonas aeruginosa as “critical priority pathogens” due to the prevalence of resistant clinical isolates, poor treatability of infections, and high mortality rates. Antibiotic discovery for P. aeruginosa has proven to be the most challenging, as drug accumulation inside these bacteria is even less likely than for other Gram-negative species. This lower permeability of compounds in P. aeruginosa is in part due to the absence of non-specific porins, such as OmpF in E. coli, which facilitate the diffusion of small, hydrophilic compounds across the outer membrane. Instead, P. aeruginosa possesses ~40 monomeric, substrate-specific channels for nutrient transport with size restrictions of ~200 Da. Additionally, P. aeruginosa can express a broad range of tripartite efflux pumps, resulting in highly efficient antibiotic efflux. This combination confers high intrinsic resistance to many antibacterials, including some that are commonly used to treat infections caused by other Gram-negative pathogens; for example, while antibiotics such as chloramphenicol, tetracyclines, and trimethoprim/sulfamethoxazole have clinical activity against other Gram-negative organisms, they are not effective against P. aeruginosa. Notably, the lack of efficacy of these drugs is attributed to poor intracellular accumulation in clinical isolates of P. aeruginosa, not to a lack of target engagement. This is also true for a host of antibiotics (e.g., erythromycin, linezolid, fusidic acid, rifampin, etc.) that are ‘Gram-positive-only’ and have no activity in P. aeruginosa or other Gram-negative pathogens.

The identification of physicochemical properties that promote intracellular compound accumulation would greatly aid in the development of new anti-pseudomonal antibiotics. For example, an understanding of compound accumulation in E. coli has led to actionable guidelines that have been used to build accumulation and/or Gram-negative antibacterial activity into multiple different classes of compounds. However, despite important retrospective and prospective analyses of antibiotic activity and accumulation in P. aeruginosa, general rules for compound accumulation in this bacterium have remained elusive. And, with multiple sources of potential uptake and efflux in P. aeruginosa, the possibility for discovery of generalizable accumulation guidelines for P. aeruginosa has been questioned.

With resistance rampant and the toxicity of certain active classes (e.g., aminoglycosides and polymyxins) significant, novel antibacterials for P. aeruginosa infections are an area of significant need.

### SUMMARY

With the goal of developing a predictive model for compound accumulation in P. aeruginosa, here we utilize a whole-cell accumulation assay to assess the ability of 345 non-antibiotic, diverse, small molecules to accumulate in this pathogen. Hundreds of physicochemical features were calculated for each compound, and a random forest classification model was trained to help understand the relationship between these chemical traits and compound accumulation. Through this process, factors related to positive charge and hydrogen-bond donor surface area were found to be important, with ~80% of compounds meeting the appropriate cut-offs accumulating in P. aeruginosa. This ultimately led to design principles that were used to understand previous antibiotic activity trends and to the de novo design of a Gram-negative active version of fusidic acid. Leveraging non-porin mediated pathways appears to be a general means to circumvent the accumulation barrier in P. aeruginosa, with important implications for the design of novel antibiotics.

Accordingly, this disclosure provides a compound of Formula I

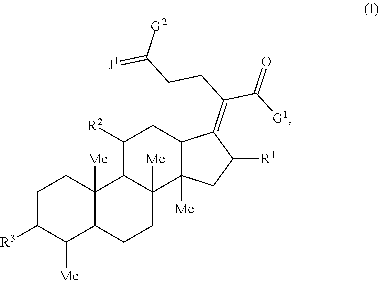

or a salt thereof;
wherein
   G¹ is —O—N═CR⁴R⁵, NR⁶R⁷, or OR⁸;
   G² is H, halo, OH, —(C₁-C₆)alkyl, —O(C₁-C₆)alkyl, or NR^{a}R^{b};
   J¹ is CR^{c}R^{d}, O, or absent;
      R^{a} and R^{b} are each independently H, —(C₁-C₆)alkyl, or —(C₃-C₆)cycloalkyl;
      R^{c} and R^{d} are each independently —(C₁-C₆)alkyl, —(C₃-C₆)cycloalkyl, H, halo, aryl, or
   (C₀-C₅)R^{e} wherein R^{c} is OH, —O(C₁-C₆)alkyl, or —C(═O)O(C₁-C₆)alkyl; or
   R^{c} and R^{d} taken together form a cycloalkyl or heterocycloalkyl;
   R¹ is —OC(═O)(C₁-C₆)alkyl, H, methyl, ethyl, hydroxy, methoxy, ethoxy, or amino;
   R² and R³ are each independently hydroxy, H, methyl, ethyl, methoxy, ethoxy, amino, or —OC(═O)(C₁-C₆)alkyl;
   R⁴ is —CH₂R⁹, aryl, heteroaryl, or alkyl, wherein R⁹ is N-polyaminoalkyl, N-monoaminoalkyl, N-heterocycloalkyl, amino, azido, halo, aryl, or heteroaryl;
   R⁵ is NR^{a}R^{b}, —(C₁-C₆)alkyl, or H; or
   R⁴ and R⁵ taken together from a heterocycloalkyl;
   R⁶ is alkylpolyamine or alkylamine;
   R⁷ is H or —(C₁-C₆)alkyl; and
R⁸ is alkylenecarbamate or alkylenecarbonate.

This disclosure also provides a method of antimicrobial treatment comprising administering to a subject in need thereof a therapeutically effective amount of a compound disclosed herein or a pharmaceutically acceptable salt thereof, thereby killing or inhibiting the growth of at least a portion of a plurality of bacteria in the subject.

The invention provides novel compounds of Formulas I-IV, intermediates for the synthesis of compounds of Formulas I-IV, as well as methods of preparing compounds of Formulas I-IV. The invention also provides compounds of Formulas I-IV that are useful as intermediates for the synthesis of other useful compounds. The invention provides for the use of compounds of Formulas I-IV for the manufacture of medicaments useful for the treatment of bacterial infections in a mammal, such as a human.

The invention provides for the use of the compositions described herein for use in medical therapy. The medical therapy can be treating bacterial infections, for example, Enterobacteriaceae, Acinetobacter baumannii, or Pseudomonas aeruginosa. The invention also provides for the use of a composition as described herein for the manufacture of a medicament to treat an infection in a mammal, for example, a gram-negative bacterial infection in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-D. Assessment of antibiotic controls in the P. aeruginosa accumulation assay and assessment of eNTRy rules predictability in P. aeruginosa. a) Inactive and covalently modified antibiotics show low levels of accumulation in P. aeruginosa PAO1, while active antibiotics are high accumulating compounds. Statistically significant accumulation over the average of low-accumulating controls is indicated with asterisks (***P<0.001). Statistical significance was determined using a two-sample Welch's t-test (one-tailed test, assuming unequal variance). b) Low-accumulating antibiotics show an increase in accumulation with treatment of 8 μg/mL of the permeabilizer polymyxin B nonapeptide (PMBN). Statistically significant accumulation differences for low accumulating compounds in permeabilized and non-permeabilized PAO1 are indicated with asterisks (***P<0.001). c) Efflux substrates chloramphenicol and valnemulin show increased accumulation in the efflux pump knockout strain, P. aeruginosa PAA6, whereas non-substrate vancomycin shows no significant increase. Statistically significant accumulation differences for low accumulating compounds in wild-type P. aeruginosa PAO1 and efflux deficient P. aeruginosa are indicated with asterisks (n.s. not significant, ***P<0.001). d) The influence of globularity and rotatable bonds on compound accumulation in P. aeruginosa PAO1 versus E. coli MG1655 for 40 primary amines. Low globularity and low rotatable bonds are predictive for ~₈₀% of compounds tested in E. coli, but only ~₅₀% for P. aeruginosa. Structures of all compounds and the data for E. coli is taken from Richter and co (Nature 2017, 545 (7654), 299-304). For all figure panels, the s.e.m is reported for accumulation values and compounds were tested in biological triplicate.

FIG. 2A-B. Importance of ClogD₇₄, hydrogen bond donor ability, and positive charge for accumulation in P. aeruginosa PAO1. a) A set of 345 compounds, including 240 primary amines, was evaluated for accumulation in P. aeruginosa PAO1. This data set includes the 67 compounds from FIG. 1e. All accumulating compounds have a positive charge. b) Analysis of 240 primary amine containing compounds, correlation of formal charge and vsa_don with compound accumulation in P. aeruginosa PAO1. In this analysis, compounds with vsa_don>23 and either Q_vsa_PPos>80 or formal charge >0.98 were most likely to accumulate. >80% of compounds that met these criteria were accumulators, shown in the grey box. 113 compounds met the criteria, and 92 of them were accumulators, while 21 were non-accumulators. 127 compounds did not meet the criteria, and 55 of them were accumulators and 72 were non-accumulators.

FIG. 3A-B. a) A subset of compounds (21-47) tested in a strain of P. aeruginosa PA14 with all 40 putative porins knocked out (PA14 A40) shows minimal accumulation differences relative to the parental strain PA14, suggesting a porin-independent mode of uptake. b) The same subset of compounds showed a statistically significant decrease in accumulation upon co-treatment with 1 mM MgCl₂, suggesting self-promoted uptake as the primary mode of entry for these compounds. The same PA14 data is used in FIG. 3a and FIG. 3b. Compounds were tested in biological triplicate. The s.e.m is reported for accumulation values.

FIG. 4. Six examples where an antibiotic derivative is more active (>4-fold) against P. aeruginosa than the parent. Analysis shows that the derivatives with improved antibacterial activity meet the vsa_don and charge requirements for accumulation in P. aeruginosa (as shown by the grey box). Biochemical activity against the target is approximately the same for the compound pairs (Table 6), suggesting that the improved antibiotic activity against P. aeruginosa is due to an increase in accumulation, which is indeed the case for the tetracycline/tigecycline pair (FIG. 1a).

FIG. 5A-C. Development of PA-active Fusidic acid (FA) derivative. a) FA does not meet the predictive guidelines for accumulation in P. aeruginosa, while FA polyamine and FA prodrug both fit the described physicochemical properties, highlighted in the grey box. b) Consistent with the property prediction, FA does not accumulate in P. aeruginosa PAO1, while both FA polyamine and FA prodrug show >30× higher accumulation levels. As the prodrug hydrolyzes under assay conditions, the accumulation of FA is reported for FA prodrug. c) FA prodrug possesses 64-256× improved activity relative to FA against a panel of 75 clinical isolates of P. aeruginosa. MICs were performed according to the CLSI guidelines in biological triplicate. Accumulation units are reported in nmol/10¹² CFUs. All compounds were tested in biological triplicate. Error bars represent s.e.m. for accumulation values.

FIG. 6. Comparison of species-specific accumulation trends. In the expanded set of primary amines, many compounds that fit the eNTRy rules do not accumulate in P. aeruginosa PAO1, and numerous compounds that do not fit the eNTRy rules do accumulate in P. aeruginosa PAO1. Low globularity and low rotatable bonds are predictive for ~80% of compounds tested in E. coli MG1655, but only 41% of compounds in P. aeruginosa PAO1. Compounds with poor amine steric accessibility, low amphiphilic moment, and multiple charges were removed from this analysis; 154 compounds total are included in the plots.

FIG. 7A-F. a) Random forest prediction model results on data set of all 240 primary amine structures. ROC plot with 10 repeated cross-validations in the training classification models. b) Relative importance of top 15 descriptors for all 240 primary amines. c) Random forest prediction model results on data set of 50 highest primary amine accumulators and 50 lowest primary amine accumulators (100 amines total). d) Relative importance of top 15 descriptors for 100 primary amines. e) Random forest prediction model results on data set of 30 highest primary amine accumulators and 30 lowest primary amine accumulators (60 amines total). f) Relative importance of top 15 descriptors for 60 primary amines. Formal charge (h_pavgQ) and hydrogen bond surface area (vsa_don) are boxed in black. Early iterations of the model with the incomplete compound library always identified these properties within the top 15 most important.

FIG. 8. Distribution of accumulation values in P. aeruginosa vs. E. coli for compounds that accumulate in both bacterial strains (131 compounds plotted). Accumulation levels were lower on average in P. aeruginosa PA01 relative to E. coli MG1655.

FIG. 9. Accumulation in other strains of P. aeruginosa. Accumulation of antibiotic controls in three P. aeruginosa strains. Accumulation is consistent with antibacterial activity reported in Table 4.

FIG. 10A-B. Mode of uptake and membrane interactions of FA prodrug. a) FA prodrug and gentamicin both permeabilize the outer membrane of P. aeruginosa PAO1 to the membrane impermeable fluorophore NPN at a 10-minute time point. Upon entry to the cell and interacting with the lipid bilayer, NPN has a significant increase in fluorescence. b) FA prodrug leads to inner membrane depolarization in P. aeruginosa PAO1, quantified using the potentiometric dye DiSC₂, while treatment with Fusidic acid shows no effect.

### DETAILED DESCRIPTION

Gram-negative antibiotic development has been hindered by a poor understanding of the types of compounds that can accumulate within these bacteria, and the presence of efflux pumps and substrate-specific outer membrane porins in Pseudomonas aeruginosa render this pathogen particularly impermeable. As a result, there are few antibiotic options for P. aeruginosa infections, and its many highly specific porins has made the prospect of discovering general accumulation guidelines seem unlikely. Here, we assess the whole-cell accumulation of 345 diverse compounds in 15 P. aeruginosa and E. coli. While certain positively charged compounds were demonstrated to permeate both bacterial species, P. aeruginosa is more restrictive as compared to E. coli. Further computational analysis identified distinct physicochemical properties of small molecules that are specifically correlated with P. aeruginosa accumulation, such as formal charge, positive polar surface area, and hydrogen bond donor surface area. Surprisingly, mode of uptake studies revealed that most small molecules permeate P. aeruginosa using a porin-independent pathway, thus enabling discovery of general P. aeruginosa accumulation trends with important implications for future antibiotic development. These discoveries were then applied to expand the spectrum of activity of a Gram-positive-only antibiotic, fusidic acid, into a version that demonstrates a dramatic (256-fold) improvement in antibacterial activity against P. aeruginosa including dozens of clinical isolates. We anticipate that these discoveries will facilitate the design and development of high-permeating antipseudomonals.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability, necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, +10%, +20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults orjuveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at a carbon atom (for alkenylene) or two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents RX where RX is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “IC₅₀” is generally defined as the concentration required to inhibit a specific biological or biochemical function by half, or to kill 50% of the cells in a designated time period, typically 24 hours.

### Embodiments of the Technology

This disclosure provides a compound of Formula I:

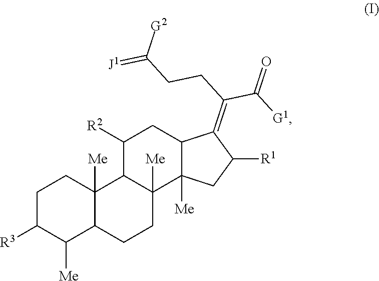

or a salt thereof;
wherein
   G¹ is —O—N═CR⁴R⁵, NR⁶R⁷, or OR⁸;
   G² is H, halo, OH, —(C₁-C₆)alkyl, —O(C₁-C₆)alkyl, or NR^{a}R^{b};
   J¹ is CR^{c}R^{d}, O, or absent;
      R^{a} and R^{b} are each independently H, —(C₁-C₆)alkyl, or —(C₃-C₆)cycloalkyl;
      R^{c} and R^{d} are each independently —(C₁-C₆)alkyl, —(C₃-C₆)cycloalkyl, H, halo, aryl, or —(C₀-C₅)R^{e} wherein R^{c} is OH, —O(C₁-C₆)alkyl, or —C(═O)O(C₁-C₆)alkyl; or
R^{c} and R^{d} taken together form a cycloalkyl or heterocycloalkyl;
   R¹ is —OC(═O)(C₁-C₆)alkyl, H, methyl, ethyl, hydroxy, methoxy, ethoxy, or amino;
   R² and R³ are each independently hydroxy, H, methyl, ethyl, methoxy, ethoxy, amino, or —OC(═O)(C₁-C₆)alkyl;
   R⁴ is —CH₂R⁹, aryl, heteroaryl, or alkyl, wherein R⁹ is —(NH(CH₂)₃)₃NH₂, N-polyaminoalkyl, N-monoaminoalkyl, N-heterocycloalkyl, amino, azido, halo, aryl, or heteroaryl;
   R⁵ is NR^{a}R^{b}, —(C₁-C₆)alkyl, or H; or R⁴ and R⁵ taken together from a heterocycloalkyl;
   R⁶ is alkylpolyamine or alkylamine;
   R⁷ is H or —(C₁-C₆)alkyl; and
      R⁸ is alkylenecarbamate or alkylenecarbonate.

In some embodiments, the compound is represented by Formula II:

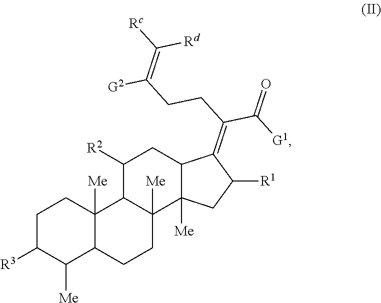

or a salt thereof.

In some embodiments, —(C₁-C₆)alkyl is C₁alkyl, C₂alkyl, C₃alkyl, C₄alkyl, C₅alkyl, or C₆alkyl. In some embodiments, —(C₃-C₆)cycloalkyl is C₃cycloalkyl, C₄cycloalkyl, C₅cycloalkyl, or C₆cycloalkyl. In some embodiments, N-polyaminoalkyl is —(NR^{a}(CH₂)ₓ)_{y}NR^{a}R^{b} wherein R^{a} and R^{b} are as defined above, x is 2-6, and y is 1-6. In some embodiments, alkylpolyamine is —((CH₂)ₓNR^{a})_{y}(CH₂)ₓNR^{a}R^{b} wherein R^{a} and R^{b} are as defined above, x is 2-6, and y is 1-6.

In some embodiments, the compound is represented by Formula III:

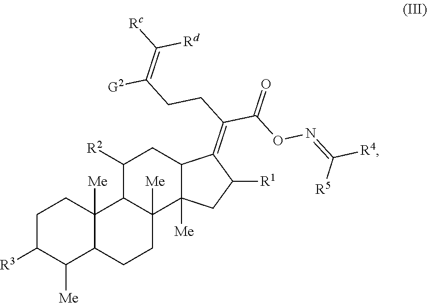

or a salt thereof.

In some embodiments, the compound is represented by Formula IV:

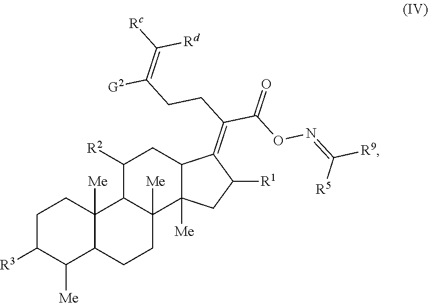

or a salt thereof.

In various embodiments, G¹ is —O—N═CR⁴R⁵ or G¹ is R⁹. In various embodiments, R⁴ is —CH₂R⁹. In various embodiments, J¹ is CR^{c}R^{d}. In various embodiments, R^{c} and R^{d} are each independently —(C₁-C₆)alkyl. In various embodiments, G² is H. In various embodiments, R⁵ is NR^{a}R^{b}. In various embodiments, R⁹ is —(NR^{a}(C₂-C₆)NR^{b})ₘ— wherein m is an integer from 1 to 6.

In various embodiments, R⁹ is NH₂, NHCH₃, —NHCH₂CH₂NH₂, —NHCH₂CH₂CH₂NH₂, —NHCH₂CH₂CH₂CH₂NH₂, —(NHCH₂CH₂)₂NH₂, —(NHCH₂CH₂CH₂)₂NH₂, —(NHCH₂CH₂CH₂CH₂)₂NH₂, —(NHCH₂CH₂)₃NH₂, —(NHCH₂CH₂CH₂)₃NH₂, —(NHCH₂CH₂CH₂CH₂)₃NH₂, —(NHCH₂CH₂)₄NH₂, —(NHCH₂CH₂CH₂)₄NH₂, —(NHCH₂CH₂CH₂CH₂)₄NH₂, —N(CH₂CH₂)₂NH, or NHCH₂Ph wherein Ph is optionally substituted with amino or halo.

In some embodiments, the compound is:

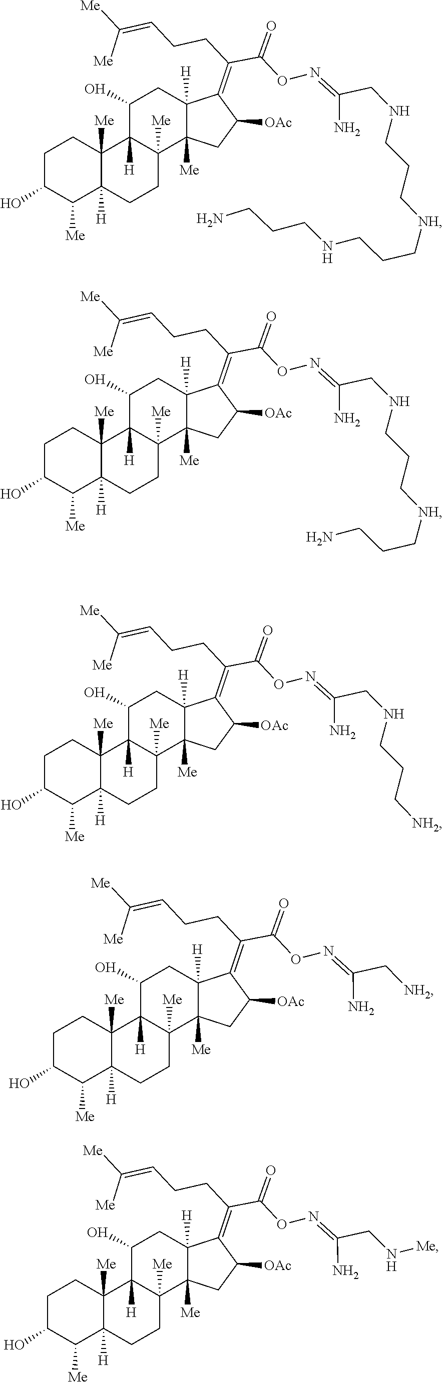

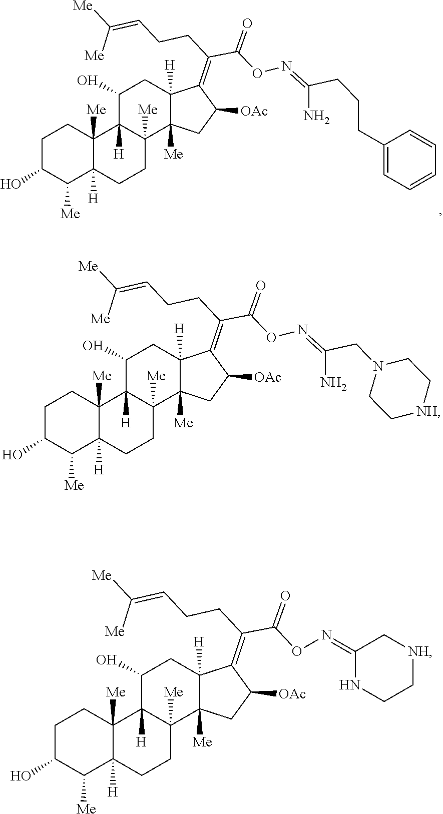

or a salt thereof.

In some embodiments, the compound is:

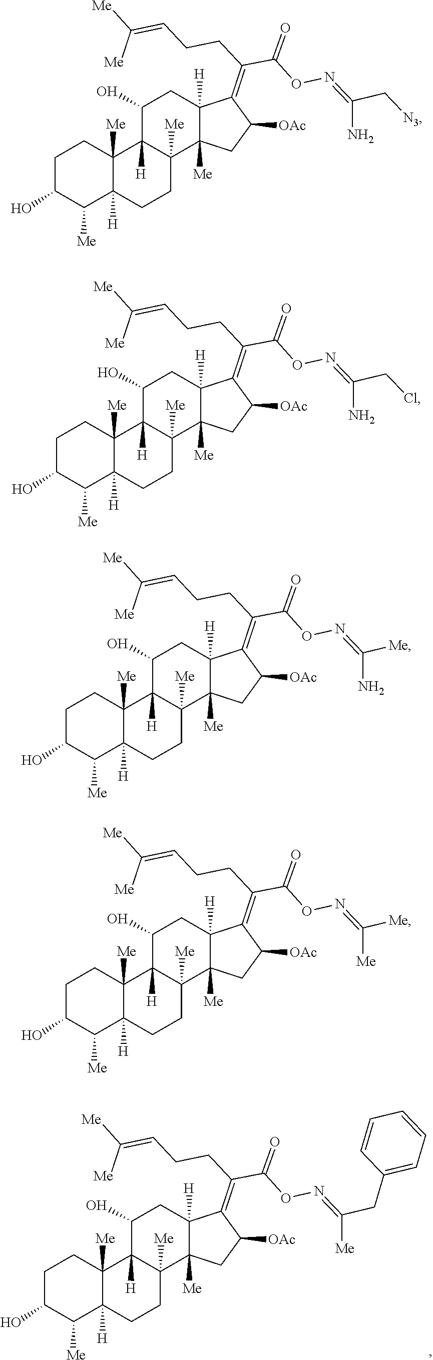

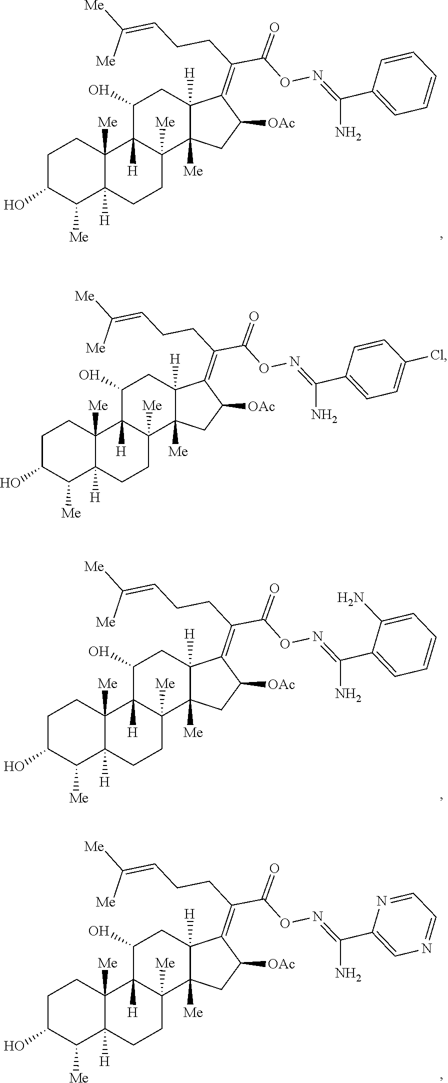

or a salt thereof.

In some embodiments, the compound is:

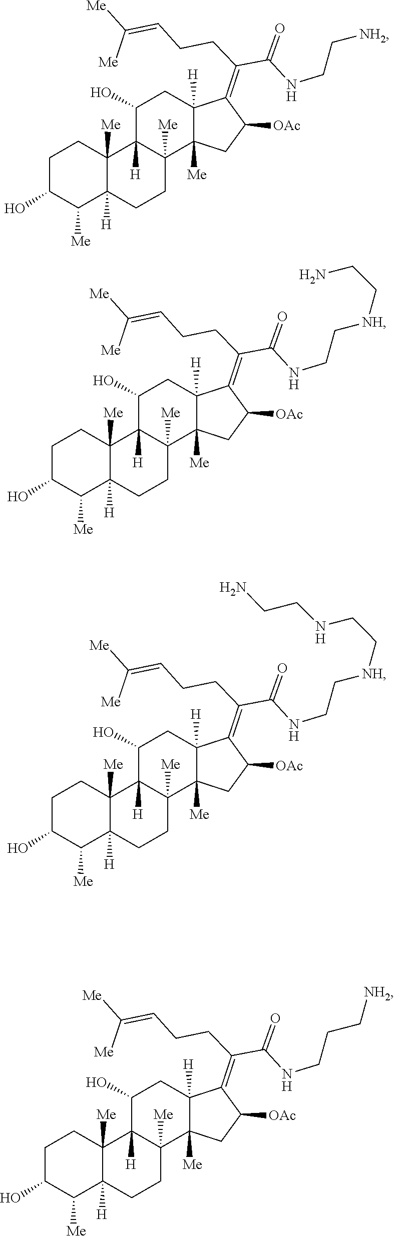

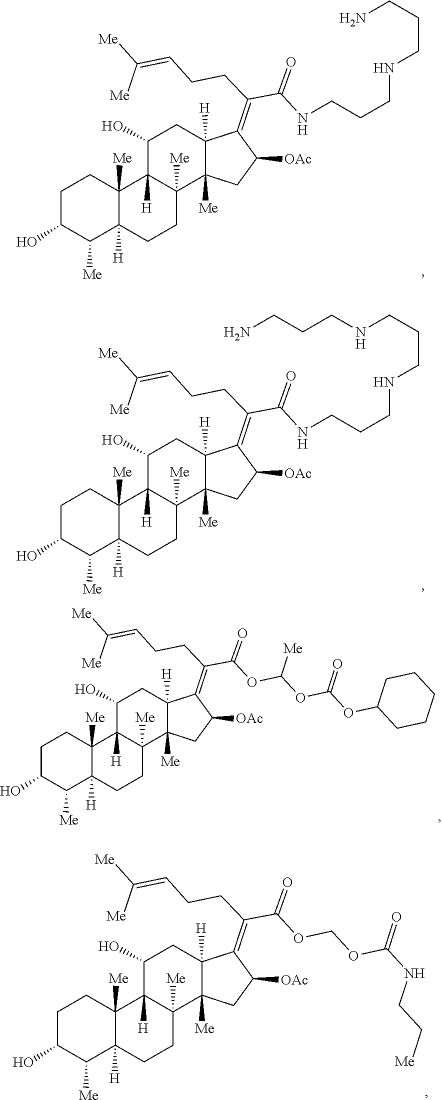

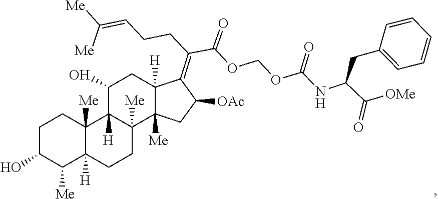

or a salt thereof.

Also, this disclosure provides a pharmaceutical composition comprising a compound disclosed herein and a pharmaceutically acceptable excipient.

Additionally, this disclosure provides a method of antimicrobial treatment comprising administering to a subject in need thereof a therapeutically effective amount of a composition or compound disclosed herein, or a pharmaceutically acceptable salt thereof, thereby killing or inhibiting the growth of at least a portion of a plurality of bacteria in the subject.

In various embodiments, the bacteria is Gram-negative bacteria. In various embodiments, the bacteria is Pseudomonas aeruginosa, Enterobacteriaceae, Acinetobacter baumannii, or a combination thereof.

In various embodiments, the bacteria is Acinetobacter, anthrax-causing bacteria, Bacilli, Bordetella, Borrelia, botulism-causing bacteria, Brucella, Burkholderia, Campylobacter, Chlanydia, cholera-causing bacteria, Clostridium, Conococcus, Cowynebacterium, diptheria-causing bacteria, Enterobacter, Einterococcus, Erwinia, Escherichia, Francisella, Haenophilus, Heliobacter, Klebsiella, Legionella, Leptospira, leptospirosis-causing bacteria, Listeria, Lyme's disease-causing bacteria, meningococcus, Mycobacteriun, Mycoplasma, Neisseria, Pasteurella, Pelobacter, plague-causing bacteria, Pneurnonococcus, Proteus, Pseudomonas, Rickettsia, Salmonella, Serratia, Shigella, Staphylococcus, Streptococcus, tetanus, Treponerna, Vibrio, Yersinia, Xanthononas, or a combination thereof.

In various embodiments, the compound is: (3R, 4S,5S,8S,9S,10S,11R,13R,14S,16S,Z)-17-((Z)-1,14-diamino-22-methyl-17-oxo-16-oxa-4,8,12,15-tetraazatricosa-14,21-dien-18-ylidene)-3,11-dihydroxy-4,8,10,14-tetramethylhexadecahydro-1H-cyclopenta[a]phenanthren-16-yl acetate.

### Results

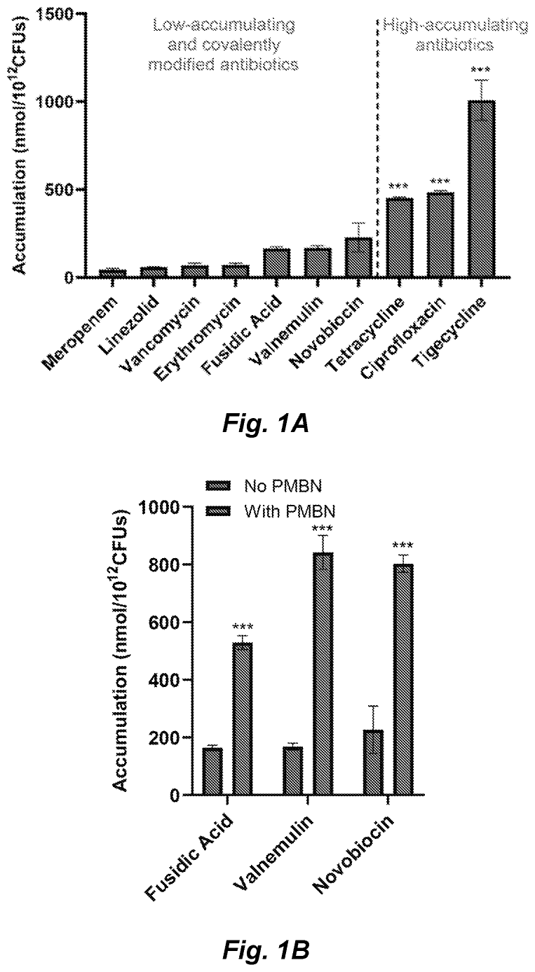

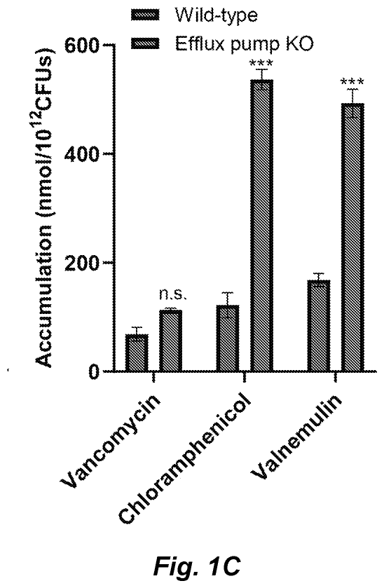

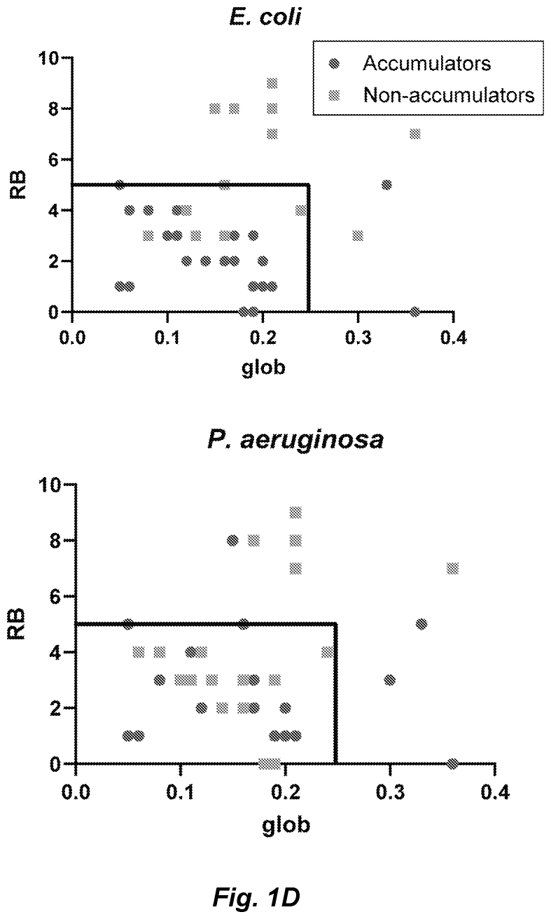

Validation of LC-MS/MS assay in P. aeruginosa. To study compound accumulation in P. aeruginosa, an LC-MS/MS-based assay was utilized. Although predominately used to examine compound accumulation in E. coli, this assay has also been used in P. aeruginosa. As a prelude to our experiments, a selection of antibiotic controls was evaluated for whole-cell accumulation in P. aeruginosa using the PAO1 strain. This panel (Table 1) included antibiotics with activity (MIC <16 g/mL) against P. aeruginosa in culture, such as tetracycline, ciprofloxacin, and tigecycline, and antibiotics inactive against P. aeruginosa (MIC>256 μg/mL), including linezolid, vancomycin, erythromycin, fusidic acid, valnemulin, and novobiocin. Meropenem was also included; as an antibiotic with a covalent mechanism of action, it would not be expected to show accumulation in this whole cell accumulation assay. The inactive antibiotics and meropenem all showed minimal accumulation in PAO1, whereas the active antibiotics all demonstrated statistically significant levels of accumulation relative to the inactive compounds (FIG. 1a). To further evaluate this assay in P. aeruginosa, bacteria were treated with the membrane permeabilizer polymyxin B nonapeptide (PMBN). The low-accumulating antibiotics fusidic acid, valnemulin, and novobiocin all showed a significant increase in accumulation upon membrane permeabilization, consistent with the assay not simply reporting on non-specific interactions with the outer membrane (FIG. 1b, Table 1). As an additional control, antibacterial activity and accumulation of low-accumulating antibiotics were also evaluated in a genetically modified strain ofP. aeruginosa PAOT, which has six efflux pumps knocked out (PAA6). MVIC ratios were used to identify efflux substrates (Table 2), and an increase in accumulation was observed for the antibiotics that were highly potentiated upon efflux pump knockout (valnemulin and chloramphenicol), while vancomycin, which shows no potentiation upon efflux pump knockout, showed no change in accumulation in this experiment (Figure ic).

TABLE 1
MICs of antibiotic controls used in the accumulation assay
against WT P. aeruginosa PAO1 and with co-treatment of
8 μg/mL of PMBN. MICs were performed in MH broth per
Clinical and Laboratory Standards Institute (CLSI) guidelines.
All experiments were performed in biological triplicate.

| | PAO1 MIC | PAO1 + PMBN |
|---|---|---|
| Compound | (μg/mL) | MIC (μg/mL) |
| Covalently modified | | |
| Meropenem | 0.25 | n.d. |
| Inactive antibiotics | | |
| Linezolid | >512 | n.d. |
| Vancomycin | >512 | n.d. |
| Erythromycin | 256 | n.d. |
| Fusidic acid | >512 | 4 |
| Valnemulin | 256 | 1 |
| Novobiocin | >512 | 1 |
| Active antibiotics | | |
| Tetracycline | 16 | n.d. |
| Ciprofloxacin | 0.5 | n.d. |
| Tigecycline | 1 | n.d. |

n.d. = not determined.

TABLE 2
MIC ratios between WT P. aeruginosa PAO1 and P. aeruginosa
PAΔ6 indicate whether a compound is subject to efflux. Vancomycin
shows no potentiation in the efflux pump KO strain, indicating
it is not liable to significant efflux out of the cell, whereas
chloramphenicol and valnemulin show substantial improvements
in antibacterial activity in the efflux pump KO strain, indicating
these compounds have considerable efflux liabilities. MICs
were performed in MH or LB broth per Clinical and Laboratory
Standards Institute (CLSI) guidelines. All experiments were
performed in biological triplicate.

| | WT MIC | PAΔ6 MIC | Fold |
|---|---|---|---|
| Compound | (μg/mL) | (μg/mL) | change |
| Poor Efflux Substrate | | | |
| Vancomycin | >512 | >512 | 1 |
| Good Efflux Substrates | | | |
| Chloramphenicol | >512 | 4 | >128 |
| Valnemulin | 256 | 0.125 | 2048 |

Assessment of compound accumulation in P. aeruginosa and comparison with eNTRy rules. To identify physicochemical properties that promote accumulation in P. aeruginosa, accumulation of a collection of diverse, non-antibiotic compounds was evaluated, including natural product-like compounds generated through the Complexity-to-Diversity strategy (Nat. Chem. 2013, 5 (3), 195-202), as well as some commercially available compounds. As a major interest was defining how accumulation trends for P. aeruginosa compared to E. coli, a set of 67 compounds whose accumulation was previously assessed in E. coli was evaluated in P. aeruginosa PAO1.

In this set of 67 compounds, positive charge did appear to be correlated with compound accumulation in P. aeruginosa PAO1, with all accumulating compounds being positively charged, as was observed for this same compound set in E. coli. The importance of positive charge for accumulation in P. aeruginosa was further demonstrated via side-by-side comparisons of similar compounds differing only in the nature of the charge. As shown with a series of putative FtsZ inhibitors, amine-containing versions accumulate in PAO1 much higher than the corresponding neutral amide with the primary amine leading to the highest levels of accumulation among the set (Chart 1; 1-4); these data mirror trends seen in E. coli with the same compounds.. Other matched compound pairs (5 and 6, 7 and 8 in Chart 1), also demonstrate how a primary amine can facilitate compound accumulation in P. aeruginosa in certain contexts.

Chart 1. The influence of amines on compound accumulation in P. aeruginosa PAO1 for three different series of compounds. Accumulation units are reported in nmol/10¹² CFUs. All compounds were tested in biological triplicate.

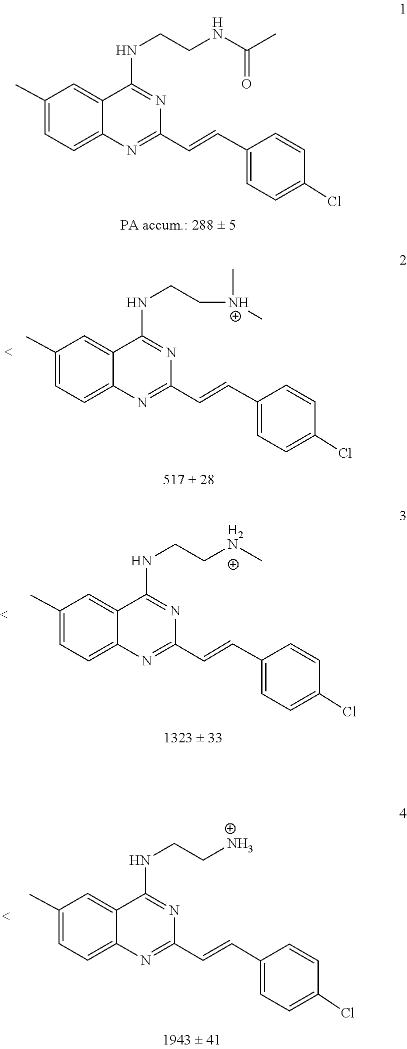

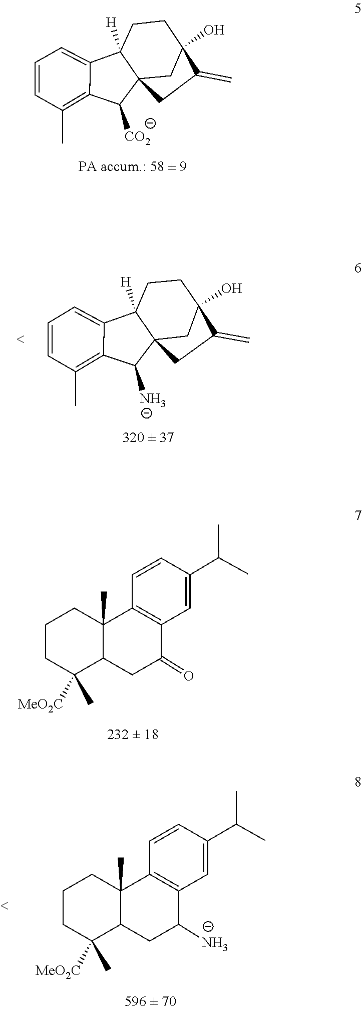

The importance of positive charge for compound accumulation, observed here for P. aeruginosa, invites comparison to the eNTRy rules (formulated for E. coli) which state that compounds with a positive charge (primary amine being best), low globularity, and a low number of rotatable bonds are most likely to accumulate in E. coli. To assess the applicability of the eNTRy rules to P. aeruginosa, data for the compounds bearing primary amines in the set of 67 compounds were plotted according to their number of rotatable bonds and globularity scores; for this analysis amines that did not meet the other criteria identified previously as important for accumulation (steric accessibility and low amphiphilic moment) were removed, leaving a total of 40 compounds. This analysis revealed that low globularity and rotatable bonds were not nearly as predictive for accumulation in P. aeruginosa compared to E. coli as these cutoffs still include many non-accumulators in P. aeruginosa, whereas for E. coli these cutoffs bin ~80% of all compounds correctly (FIG. 1e), consistent with previous results in E. coli for a large compound set. Further, in this small set, four compounds outside the eNTRy rules accumulate in P. aeruginosa (FIG. 1e). These exceptions to the eNTRy rules in both directions suggest that, besides the beneficial introduction of a primary amine, the eNTRy rules do not correctly predict compound accumulation in P. aeruginosa, with only ~50% of all compounds binned correctly in this data set.

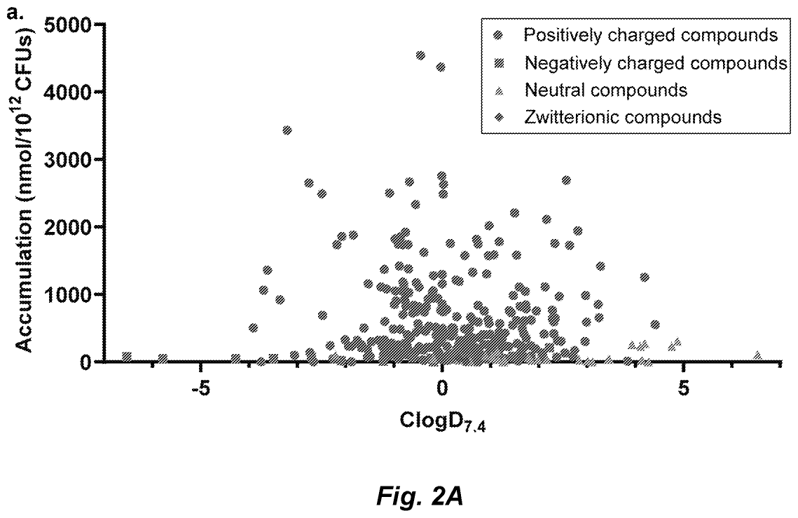

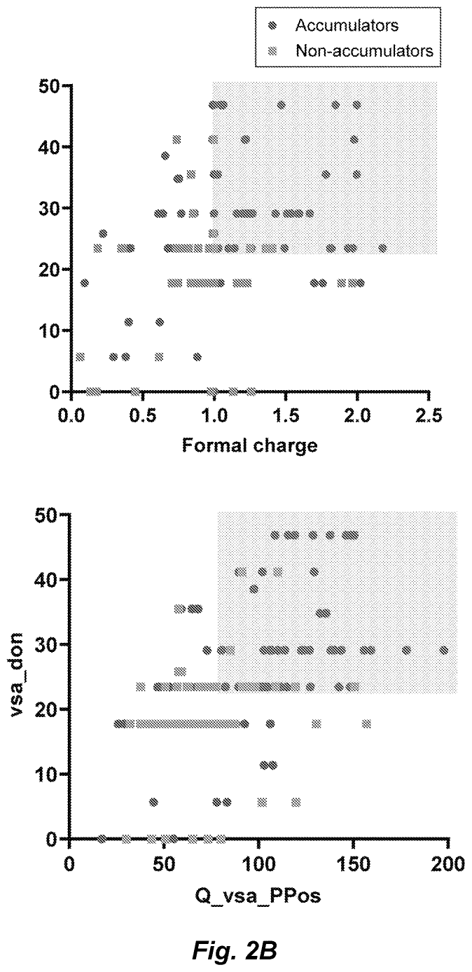

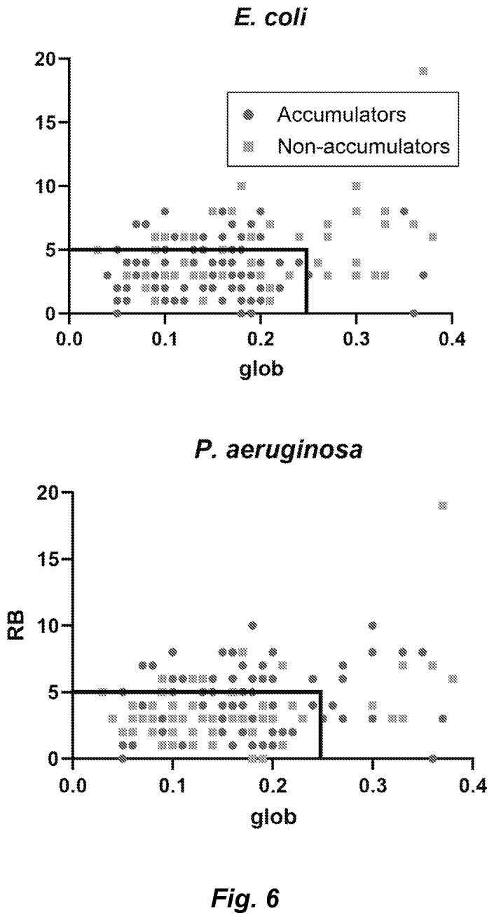

As not all compounds that possess a primary amine accumulate in P. aeruginosa, the compound test set was expanded to identify other properties that facilitate accumulation. In all, 240 primary amines and 105 other compounds with varying charge state were evaluated for accumulation in P. aeruginosa PAO1. Data gathered with this expanded collection of 345 compounds confirmed the findings from the smaller compound set. It was observed that many positively charged compounds accumulate (FIG. 2a) and accumulators span a broad range of ClogD_{7.4} values (FIG. 2a). Further, while ~80% of these new compounds meeting the eNTRy rule cutoffs accumulate in E. coli, in agreement with the original report, the number of rotatable bonds and globularity are not useful for predicting accumulation in P. aeruginosa, with only ~40% of the compounds being sorted correctly (FIG. 6). Chart 2 also shows examples of compounds that accumulate in E. coli (48-53) but not P. aeruginosa (54-56).

CHART 2
a) Compounds that accumulate in E. coli, but not in P. aeruginosa, can often be grouped
according to structural class, but no additional trends were identified. b) Compounds that
accumulate in P. aeruginosa, but not E. coli, are primarily compounds that have high rotatable
bonds, high globularity, or both. Accumulation units are reported in nmol/10¹² CFUs. All compounds
were tested in biological triplicate. The s.e.m. is reported for accumulation values.

| a. | | | |
|---|---|---|---|
| E. coli only accumulators | | | |
| Isomannide derivatives | | | |
| | 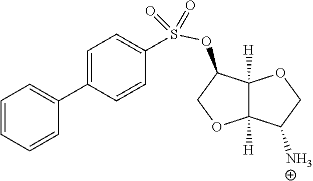 [SMILES: [NH3+][C@H]1CO[C@H]2[C@@H]1OC[C@H]2OS(=O)(=O)c1ccc(-c2ccccc2)cc1] | 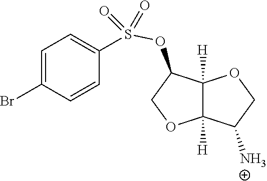 [SMILES: [NH3+][C@H]1CO[C@H]2[C@@H]1OC[C@H]2OS(=O)(=O)c1ccc(Br)cc1] | 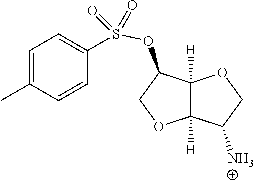 [SMILES: Cc1ccc(S(=O)(=O)O[C@@H]2CO[C@H]3[C@@H]2OC[C@@H]3[NH3+])cc1] |
| | 48 | 49 | 50 |
| PA accum .: | 282 ± 37 | 217 ± 3 | 163 ± 39 |
| EC accum .: | 1965 ± 291 | 1647 ± 108 | 877 ± 29 |
| Isoquinolone derivatives | | | |
| | 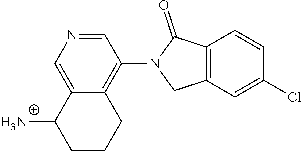 [SMILES: [NH3+]C1CCCc2c1cncc2N1Cc2cc(Cl)ccc2C1=O] | 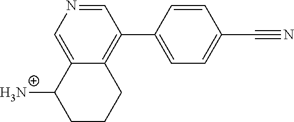 [SMILES: N#Cc1ccc(-c2cncc3c2CCCC3[NH3+])cc1] | 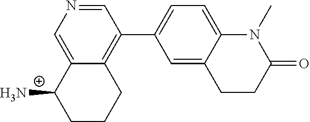 [SMILES: CN1C(=O)CCc2cc(-c3cncc4c3CCC[C@H]4[NH3+])ccc21] |
| | 51 | 52 | 53 |
| PA accum: | 222 ± 11 | 161 ± 12 | 282 ± 37 |
| EC accum.: | 500 ± 10 | 929 ± 39 | 437 ± 28 |
| b. | | | |
| P. aeruginosa only accumulators | | | |
| | High RB | High glob | High RB and glob |
| | 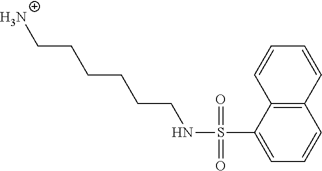 [SMILES: [NH3+]CCCCCCNS(=O)(=O)c1cccc2ccccc12] | 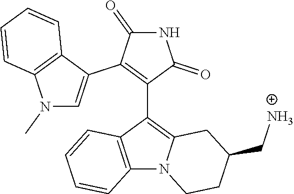 [SMILES: Cn1cc(C2=C(c3c4n(c5ccccc35)CC[C@H](C[NH3+])C4)C(=O)NC2=O)c2ccccc21] | 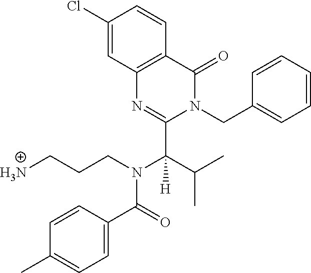 [SMILES: Cc1ccc(C(=O)N(CCC[NH3+])[C@@H](c2nc3cc(Cl)ccc3c(=O)n2Cc2ccccc2)C(C)C)cc1] |
| | 54 | 55 | 56 |
| PA accum.: | 399 ± 63 | 1193 ± 27 | 658 ± 113 |
| EC accum.: | 108 ± 17 | 17 ± 7 | 244 ± 31 |

Beyond the three eNTRy rule parameters, two other properties were previously identified as important for accumulation in E. coli, amphiphilic moment and amine steric accessibility. Consistent with the observation in E. coli, increasing amphiphilic moment (measured asvsurf A) correlated with an increase in compound accumulation in P. aeruginosa (Chart 3; 57-60). The steric accessibility of the primary amines also made a significant difference in accumulation values. Like E. coli, compounds with primary amines accumulated to a greater extent than those with secondary or tertiary amines (Chart 3; 61 and 62). Additionally, primary amines on primary carbons accumulated to a greater extent than primary amines on secondary or tertiary carbons (Chart 3; 63 and 64).

Chart 3. a) Amphiphilic moment positively correlates with accumulation in both P. aeruginosa and E. coli. b) Amine steric accessibility is important for accumulation in both P. aeruginosa and E. coli. Primary amines accumulate higher than secondary or tertiary amines in general. Similarly, primary amines on primary carbons tend to accumulate higher than primary amines on secondary carbons, which accumulate higher than primary amines on tertiary carbons. Accumulation units are reported in nmol/10¹² CFUs. All compounds were tested in biological triplicate. The s.e.m is reported for accumulation values. Strains used: E. coli MG1655, P. aeruginosa PAO1.

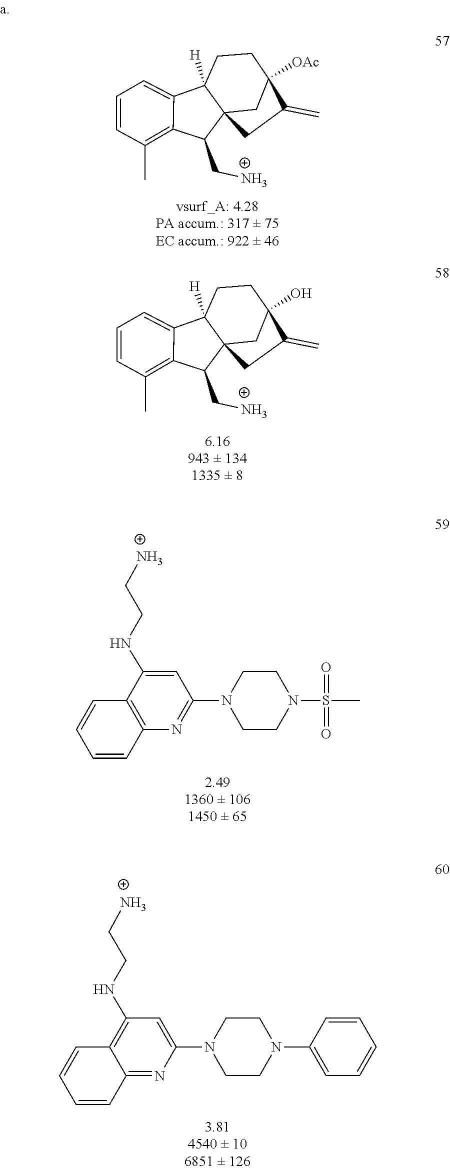 [SMILES: C=C1C[C@]23C[C@@]1(OC(C)=O)CC[C@@H]2c1cccc(C)c1[C@@H]3C[NH3+] ; C=C1C[C@]23C[C@@]1(O)CC[C@@H]2c1cccc(C)c1[C@@H]3C[NH3+] ; CS(=O)(=O)N1CCN(c2cc(NCC[NH3+])c3ccccc3n2)CC1 ; [NH3+]CCNc1cc(N2CCN(c3ccccc3)CC2)nc2ccccc12]

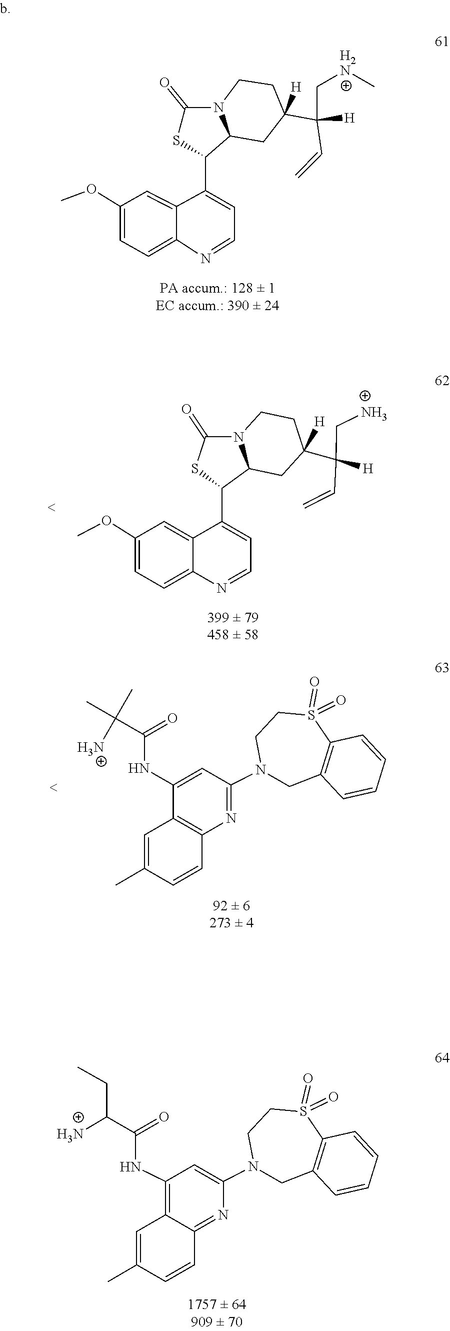

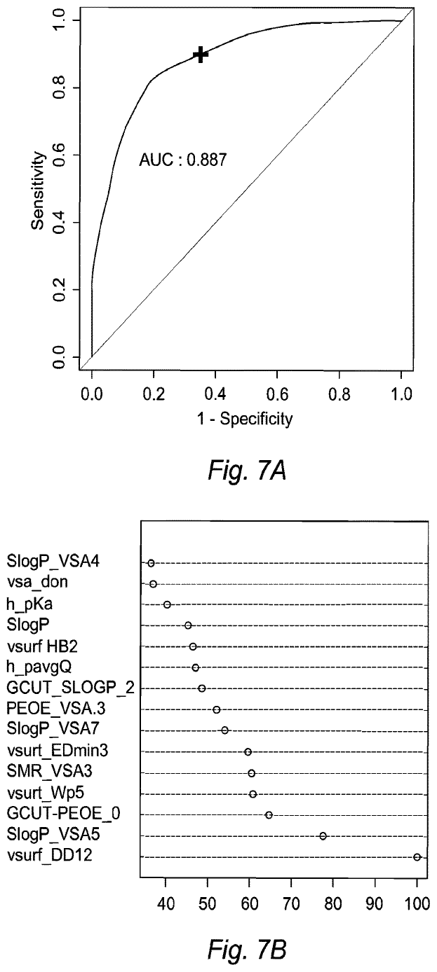

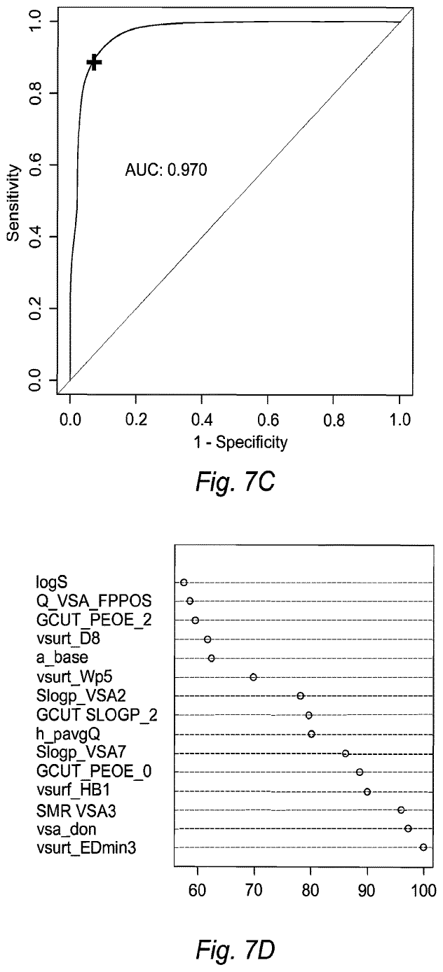

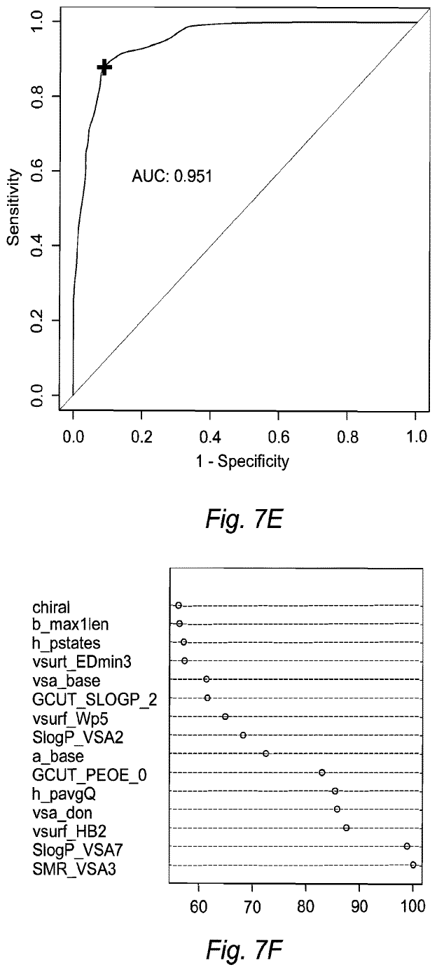

Identification of chemical features that influence compound accumulation in P. aeruginosa. While the presence of a sterically unencumbered primary amine on a compound with a high amphiphilic moment correlates with accumulation in P. aeruginosa, these traits alone are not enough to form a useful set of guidelines. Thus, we investigated other physicochemical traits that might facilitate accumulation in P. aeruginosa. For this work a cheminformatic approach was utilized, starting with the calculation of 288 physicochemical properties for each of the 240 primary amines shown in FIG. 2a. These descriptors were used to train a random forest classification model to predict compound accumulation (FIG. 7) (Nature 2017, 545 (7654), 299-304). Through this analysis, it was noted that hydrogen bond donor surface area (measured by total van der Waals surface area of hydrogen bond donors (vsa_don)) and positive charge descriptors were positively correlated with accumulation.

The most predictive descriptors associated with positive charge were positive polar surface area (Q_vsa_Ppos) and overall formal charge. While these two positive charge descriptors have commonalities, using both accounts for compounds that 1) may have a primary amine with a lower pKₐ and less localized charge, but have other partially charged functional groups that are beneficial for accumulation (low formal charge, high positive polar surface area), and 2) are smaller compounds that may be highly charged, but have lower overall surface area (high formal charge, low positive polar surface area). Compounds with vsa_don>23 and with high positive charge (Q_vsa_Ppos>80 and/or formal charge >0.98) were most likely to accumulate (grey box in FIG. 2b), with >80% (92/113) of all compounds that meet these criteria accumulating in P. aeruginosa PAO1. Side-by-side comparisons demonstrate the benefit of increasing hydrogen bond donor surface area (Chart 4; 9-12), positive polar surface area (Chart 4; 13-16), and formal charge, either through modulation of amine pKₐ (Chart 4; 17 and 18) or through modulation of the pKₐ of other ionizable atoms (Chart 4; 19 and 20).

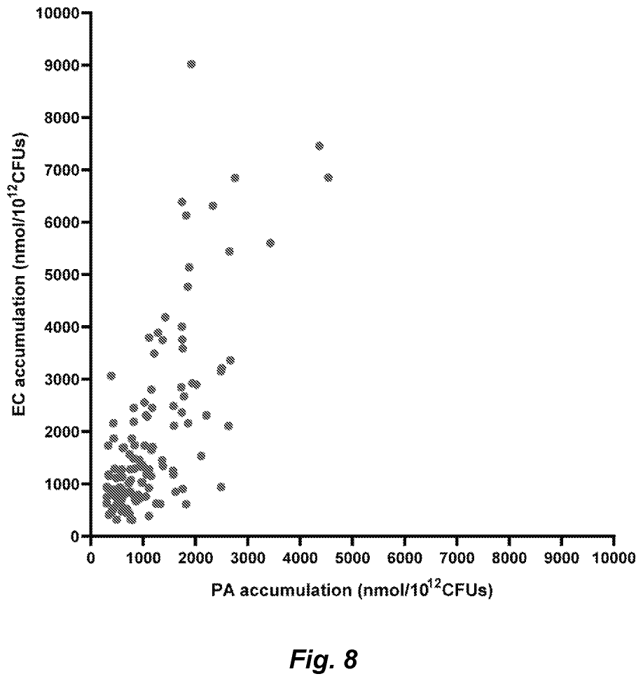

It is important to note that overall accumulation values in P. aeruginosa PAO1 are still ~50% lower on average compared to E. coli MG1655, consistent with the previously identified increased permeability barrier of P. aeruginosa relative to other Gram-negative pathogens (FIG. 8).

Chart 4. a) Increasing vsa_don leads to an increase in accumulation in P. aeruginosa. b) Increasing the positive polar surface area on a particular scaffold positively correlates with accumulation. c) Increasing the formal charge (FC) of a molecule through pKₐ modulation of amines or other ionizable atoms increases accumulation in P. aeruginosa. Accumulation units are reported in nmol/10¹² CFUs. All compounds were tested in biological triplicate. The s.e.m. is reported for accumulation values. ClogD_{7.4} was calculated using the online compound property calculation software FAFdrugs. Formal charge, vsa_don, and Q_vsa_PPos were calculated in MOE.

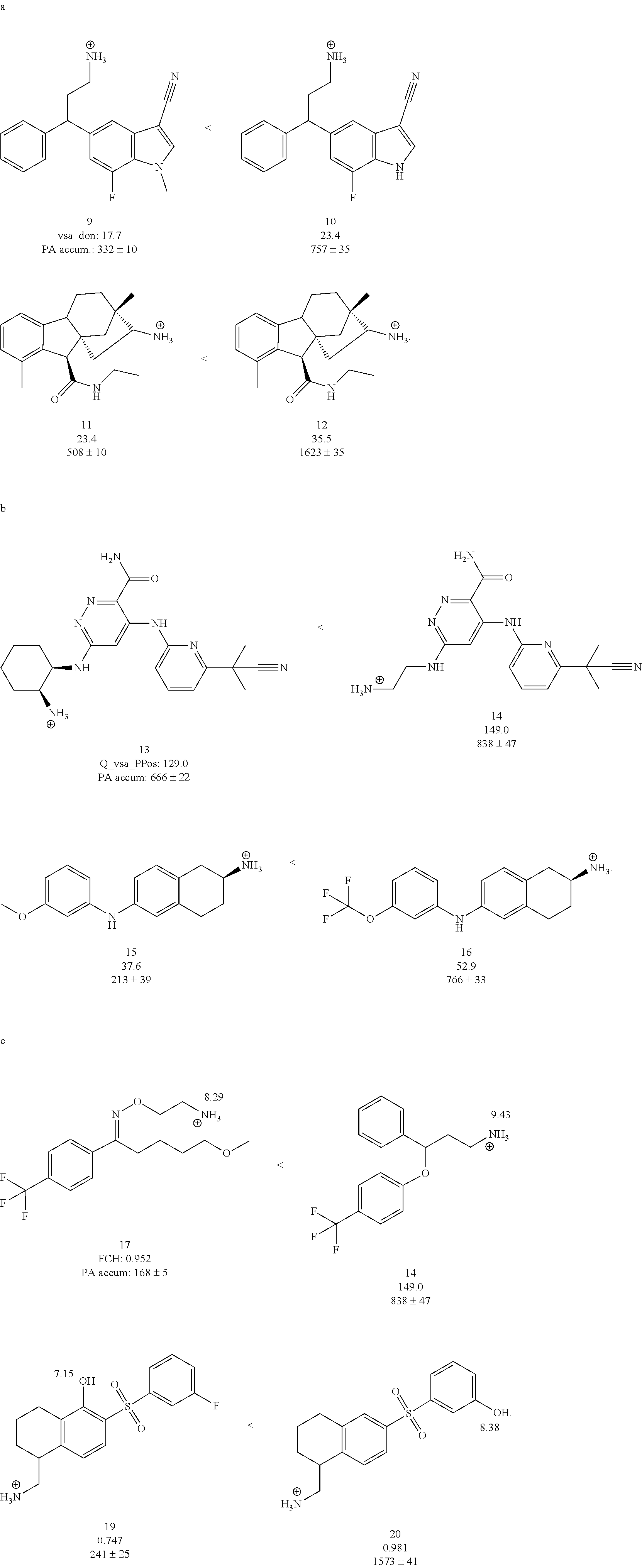

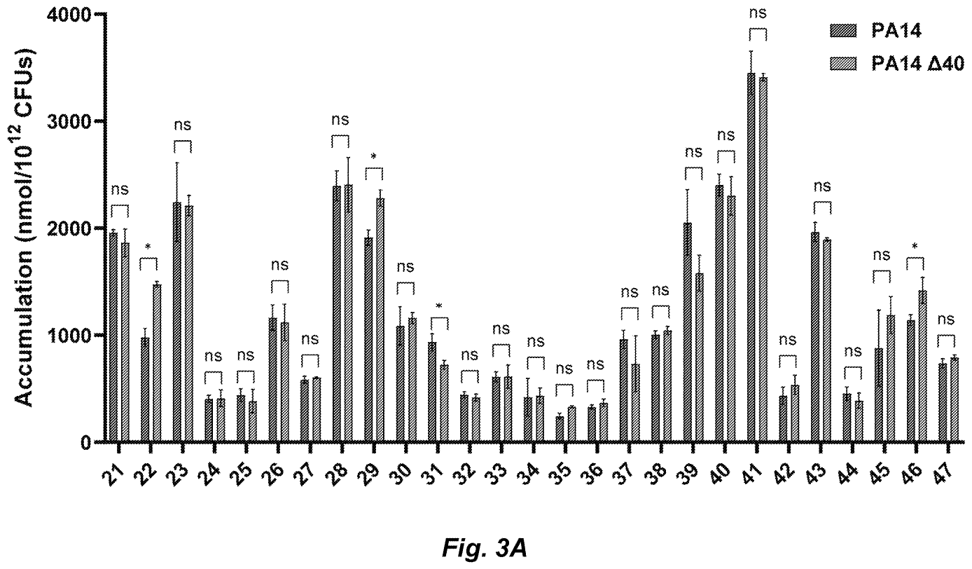

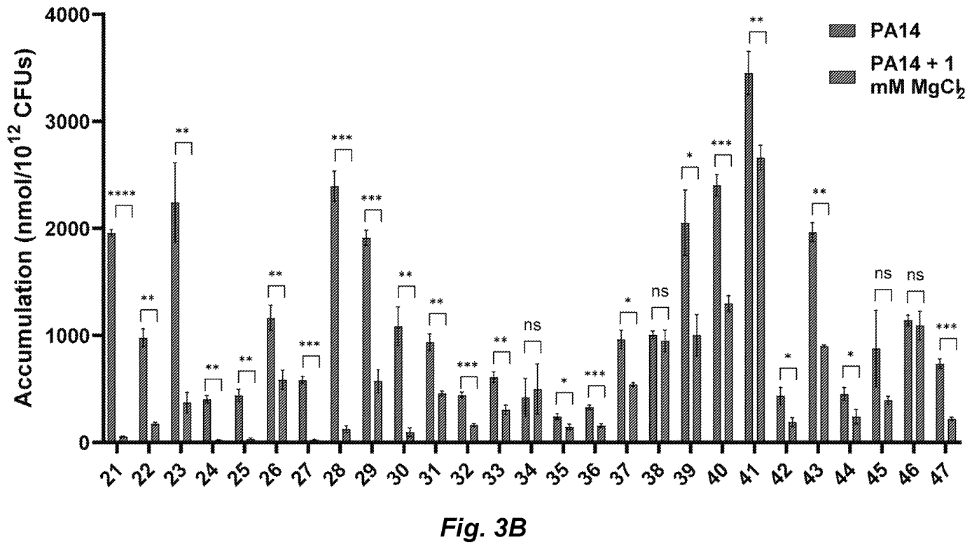

Mode of uptake. With the importance of positive charge for accumulation in P. aeruginosa established, and with a wide range of potential porins responsible for compound uptake in this organism, it was of interest to further investigate the mode of uptake for these compounds. In an attempt to identify compounds that rely on substrate-specific channels of P. aeruginosa for uptake, accumulation was assessed for a subset of compounds in a strain of P. aeruginosa that has 40 putative porins knocked out (PA14 A40) and accumulation values were compared to the parental strain, PA14. Compounds ranged in molecular weight from less than 200 to greater than 500 Da, and included monoamines, diamines, and guanidiniums. Strikingly, most compounds showed no statistically significant difference in accumulation between the two strains, indicating that even the absence of 40 porins does not significantly alter accumulation (FIG. 3a). Only one compound (31) showed a statistically significant decrease in accumulation in the PA14 A40 strain, while three compounds (22, 29, 46) had slightly higher accumulation values.

Suspecting the self-promoted uptake pathway as the mode of permeation, the same subset of compounds was evaluated for accumulation in P. aeruginosa in media supplemented with 1 mM MgCl₂. Magnesium ions compete for LPS binding sites with cationic species, and co-treatment is often used to identify compounds that use the self-promoted uptake pathway. Notably, in P. aeruginosa, monoamines, diamines, and guanidiniums showed substantially diminished accumulation in the presence on magnesium ions (FIG. 3b), suggesting a significant role for the self-promoted uptake pathway for these compounds. These accumulation results suggest that, in contrast to other Gram-negative species, P. aeruginosa does not rely on porins for most small-molecule uptake; instead, self-promoted uptake accounts for most permeation into the cell.

Accumulation of diamines. The importance of self-promoted uptake and positive charge for accumulation led us to examine the impact of multiple positive charges on accumulation. In two examples it was observed that diamines exhibited higher accumulation values relative to their monoamine comparators (Chart 5a; 65-68). Notably, while diamines containing two primary amines are almost always high accumulators (Chart 5b; 69-72), diamines with one primary amine and one secondary or tertiary amine less reliably provide an accumulation benefit in P. aeruginosa (Chart 5c; 73-76), further highlighting the impact of amine steric accessibility and hydrogen bond donor surface area on uptake.

Accumulation of other positively charged functional groups. The introduction of an amine is not always expected to be tolerated on a small molecule drug candidate; therefore, having other functional group options for improving accumulation would be beneficial. Guanidiniums and pyridiniums provide positively-charged alternatives that are more lipophilic relative to primary amines and have previously been demonstrated to improve accumulation in E. coli to differing extents. To assess the accumulation benefit of these functional groups in P. aeruginosa, a subset of 16 amine, guanidinium, and pyridinium side-by-side comparisons (48 compounds total) were evaluated for accumulation in P. aeruginosa PAOT. Guanidiniums had comparable accumulation values and classification relative to their primary amine comparators, whereas most pyridiniums led to a substantial decrease in accumulation and were not classified as accumulators (Table 3); this result matches observations in E. coli.

Chart 5. Multiple amines and alternative positive charges often aid in accumulation in P. aeruginosa PAO1. a) Diamines accumulate to significantly higher levels intracellularly in P. aeruginosa relative to their monoamine comparators. b) Diamines containing two primary amines consistently accumulate to a significant extent. c) Diamines containing one primary amine and one secondary or tertiary amine have more variable accumulation levels in P. aeruginosa, depending on hydrogen bond donor ability.

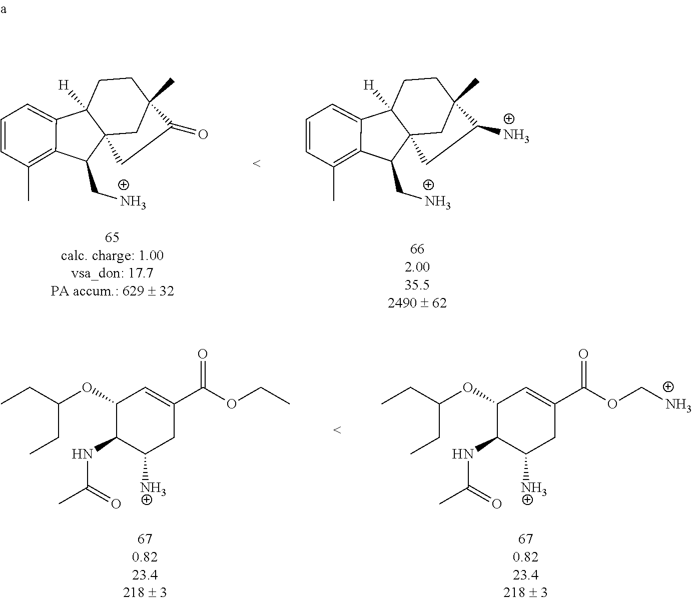 [SMILES: Cc1cccc2c1[C@H](C[NH3+])[C@@]13CC(=O)[C@@](C)(CC[C@H]21)C3 ; Cc1cccc2c1[C@H](C[NH3+])[C@@]13C[C@H]([NH3+])[C@@](C)(CC[C@H]21)C3 ; CCOC(=O)C1=C[C@@H](OC(CC)CC)[C@H](NC(C)=O)[C@@H]([NH3+])C1 ; CCC(CC)O[C@@H]1C=C(C(=O)OC[NH3+])C[C@H]([NH3+])[C@H]1NC(C)=O]

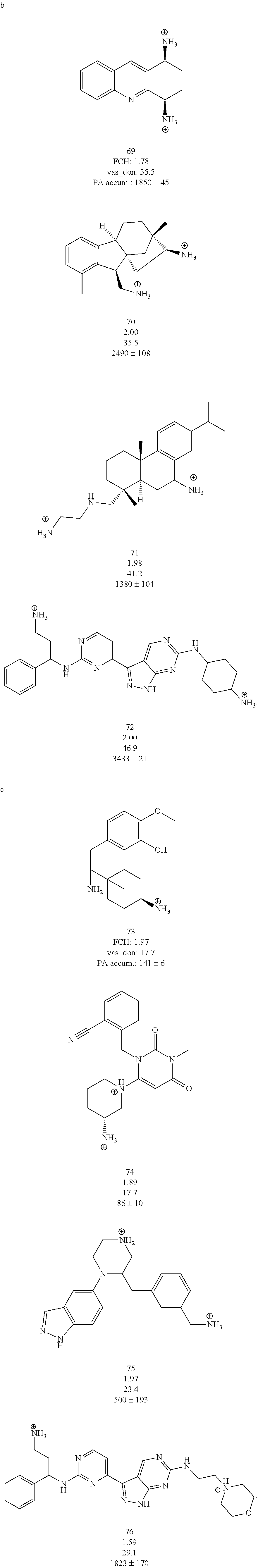

TABLE 3
a.

| Functional Group | Accumulator | Non-Accumulator | % Accumulator |
|---|---|---|---|
| Amine | 10 | 6 | 63% |
| Guanidinium | 11 | 5 | 69% |
| Pyridinium | 4 | 12 | 25% |

b.

| | | |
|---|---|---|
| 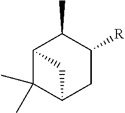 A: 1050 ± 20 G: 915 ± 68 P: 245 ± 9 | 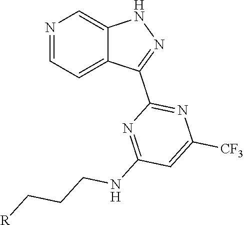 | 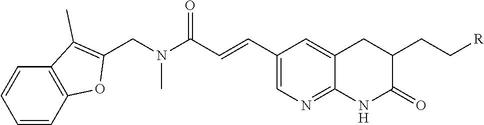 A: 650 ± 42 G: 929 ± 39 P: 120 ± 17 |
| | A: 1027 ± 49 | |
| | G: 1050 ± 65 | |
| | P: 187 ± 17 | |

a) Accumulation summary of 16 amine, guanidinium, and pyridinium containing compounds in P. aeruginosa PAO1. Compounds are classified as accumulators or non-accumulators based on statistical significance relative to the negative antibiotic controls.
b) Examples of side-by-side amine (A), guanidinium (G), and pyridinium (P) comparators and their relative accumulation values in P. aeruginosa PAO1. Amine and guanidinium comparators tend to accumulate to a very similar extent, while pyridiniums often do not accumulate to a significant extent. Accumulation units are reported in nmol/1012 CFUs. All compounds were tested in biological triplicate. The s.e.m is reported for accumulation values.

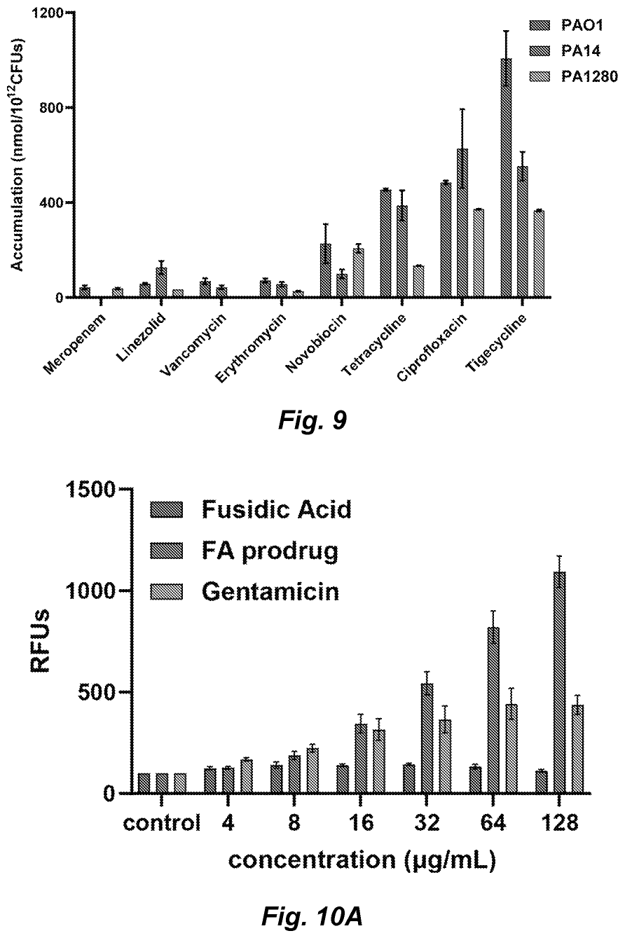

Accumulation trends across different PA strains. P. aeruginosa has a highly complex and dynamic genome, leading to significant heterogeneity between strains. To test the generality of the accumulation trends observed in P. aeruginosa PAO1, additional representative strains of P. aeruginosa were selected. P. aeruginosa PA14 is a highly virulent strain that is a part of the most common clonal group and contains pathogenicity islands in its genome that are absent in PAO1. Additionally, a clinical isolate from an acute infection, P. aeruginosa PA1280, was also evaluated. MICs for antibiotics against each of these strains were determined and intracellular accumulation was measured in the accumulation assay. Accumulation of these antibiotic controls correlated well with the activity observed (FIG. 9 and Table 4). PA1280 is slightly less susceptible to several antibiotics, which was reflected in the accumulation values.

TABLE 4
MICs of antibiotic controls in three different strains of
P. aeruginosa. MICs were performed in MH broth per Clinical
and Laboratory Standards Institute (CLSI) guidelines. All
experiments were performed in biological triplicate.

| | PAO1 MIC | PA14 MIC | PA1280 MIC |
|---|---|---|---|
| Compound | (μg/mL) | (μg/mL) | (μg/mL) |
| Covalently modified | | | |
| Meropenem | 0.25 | 0.5 | 0.5 |
| Inactive antibiotics | | | |
| Linezolid | >512 | >512 | >512 |
| Vancomycin | >512 | >512 | >512 |
| Erythromycin | 256 | 256 | 256 |
| Novobiocin | >512 | >512 | >512 |
| Active antibiotics | | | |
| Tetracycline | 16 | 8 | 32 |
| Ciprofloxacin | 0.5 | 0.125 | 0.5 |
| Tigecycline | 1 | 2 | 16 |

Additional compounds were selected for the evaluation of accumulation in PA14 and PA1280, comprising of 27 non-antibiotics with ranging levels of accumulation in PAO1. While some variance in overall values occurred, and accumulation was on average lower in PA1280, very few compounds changed classification from “accumulator” to “non-accumulator” or vice versa in either strain, suggesting that the accumulation model will be robust across different P. aeruginosa strains (Table 5).

TABLE 5
Accumulation of representative non-antibiotics in three P. aeruginosa
strains. While there is some variance in accumulation levels between
strains, high concordance of accumulation classification was observed.
Compounds are classified as accumulators or non-accumulators based on
statistical significance relative to the negative antibiotic controls.
Accumulation units are reported in nmol/10¹² CFUs. All compounds
were tested in biological triplicate. The s.e.m is reported for
accumulation values.

| | | | % Concordance |
|---|---|---|---|
| Strain | Accumulator | Non-Accumulator | with PAO1 |
| PAO1 | 14 | 13 | 100% |
| PA14 | 16 | 11 | 93% |
| PA1280 | 13 | 14 | 96% |

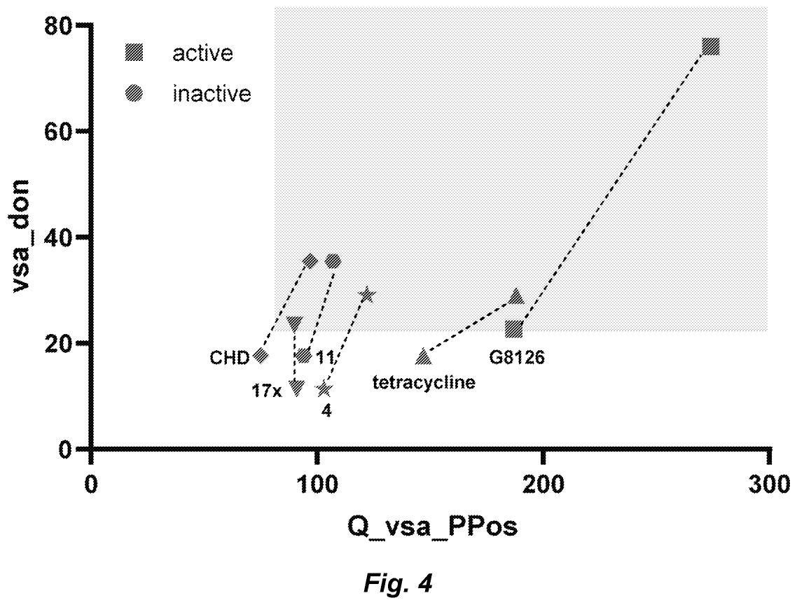

Retrospective examples. A retrospective analysis of the literature was performed to assess whether previous serendipitous incorporation of these physicochemical properties had proved beneficial in historical drug discovery campaigns. Six examples of antibiotic pairs were identified in which one derivative had poor whole-cell activity against P. aeruginosa, but another derivative was reported with improved activity. Antibiotics were selected from diverse structural classes and engage a variety of biological targets, including DNA gyrase and topoisomerase (17× and 17r; 11 and 10; 4 and 21), the ribosome (CHD and CDCHD; tetracycline and tigecycline), and the bacterial type 1 signal peptidase (G8126 and G0775). As biochemical potency and accumulation both contribute to whole-cell activity, we aimed to select side-by-side comparisons of compounds with similar target engagement. Biochemical potency and MICs in efflux pump deficient strains were used to compare target engagement (Table 6), and many of the reported pairs demonstrated similar activity in efflux pump deficient bacterial strains. The hydrogen bond donor surface area and formal charge/positive polar surface area were calculated for these antibiotic pairs. Notably, increasing hydrogen bond donor surface area to ≥23 and increasing positive polar surface area to Q_vsa_Ppos≥80 led to an increase in antibacterial activity, ranging from 4->32-fold improvements (data plotted in FIG. 4; structures and biochemical activity reported in Table 6), suggesting that increased accumulation is a significant contributor to the observed improvement in antibacterial activity. Indeed, as shown in FIG. 1a, we find tigecycline accumulates to a higher extent than tetracycline.

TABLE 6
Biological data on compounds included in the retrospective analysis comparing the
properties of PA-active compounds versus less active derivatives.
MICs reported in ug/mL. n.r. = not reported.

| | | | PA | | | |
|---|---|---|---|---|---|---|
| | | WT | efflux | WT | tolC | |
| | Compound | PA | KO | EC | EC | Enzymatic |
| Structure | ID | MIC | MIC | MIC | MIC | inhibition |
| 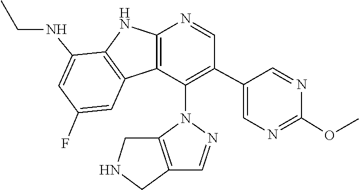 [SMILES: CCNc1cc(F)cc2c1[nH]c1ncc(-c3cnc(OC)nc3)c(-n3ncc4c3CNC4)c12] | 17x | 10 | n.r. | 0.81 | n.r. | n.r. |
| 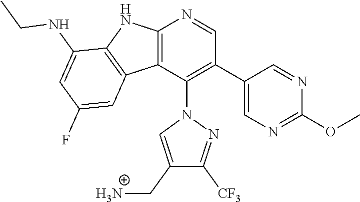 [SMILES: CCNc1cc(F)cc2c1[nH]c1ncc(-c3cnc(OC)nc3)c(-n3cc(C[NH3+])c(C(F)(F)F)n3)c12] | 17r | 1.3 | n.r. | 0.31 | n.r. | n.r. |
| 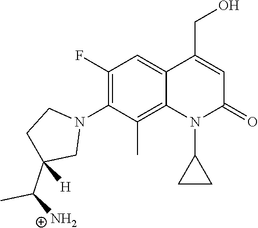 [SMILES: Cc1c(N2CC[C@@H]([C@H](C)[NH3+])C2)c(F)cc2c(CO)cc(=O)n(C3CC3)c12] | 11 | 16 | 4 | 16 | 0.25 | n.r. |
| 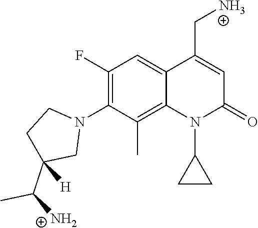 [SMILES: Cc1c(N2CC[C@@H]([C@H](C)[NH3+])C2)c(F)cc2c(C[NH3+])cc(=O)n(C3CC3)c12] | 10 | 2 | 0.5 | 1 | 0.06 | n.r. |
| 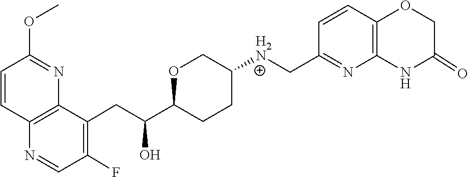 | 4 | 4 | n.r. | 0.5 | 0.004 | IC₅₀ DNA gyrase/Topo IV P. aeruginosa: 0.125/0.125 μM |
| 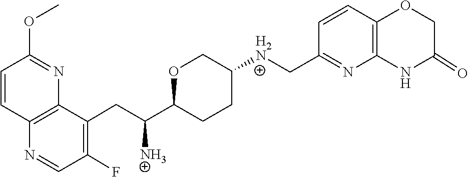 | 21 | 1 | n.r. | 0.063 | 0.008 | IC₅₀ DNA gyrase/Topo IV P. aeruginosa: 0.03/0.03 μM |
| 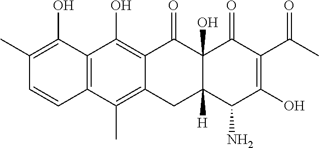 [SMILES: CC(=O)C1=C(O)[C@H](N)[C@@H]2Cc3c(c(O)c4c(O)c(C)ccc4c3C)C(=O)[C@]2(O)C1=O] | CHD | 16 | n.r. | n.r. | 0.5 | n.r. |
| 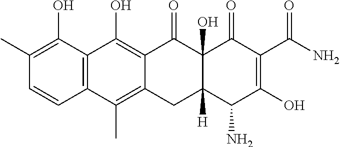 [SMILES: Cc1ccc2c(C)c3c(c(O)c2c1O)C(=O)[C@]1(O)C(=O)C(C(N)=O)=C(O)[C@H](N)[C@@H]1C3] | CDCHD | 1 | n.r. | n.r. | 0.25 | n.r. |
| 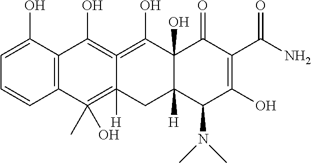 | Tetracycline | 16 | 0.25 | 1 | 0.25 | n.r. |
| 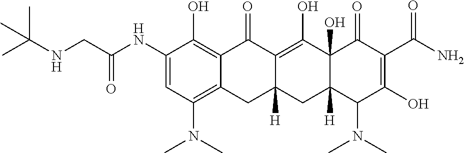 [SMILES: CN(C)c1cc(NC(=O)CNC(C)(C)C)c(O)c2c1C[C@H]1C[C@H]3C(N(C)C)C(O)=C(C(N)=O)C(=O)[C@@]3(O)C(O)=C1C2=O] | Tigecycline | 1 | 0.125 | 0.5 | 0.125 | n.r. |
| 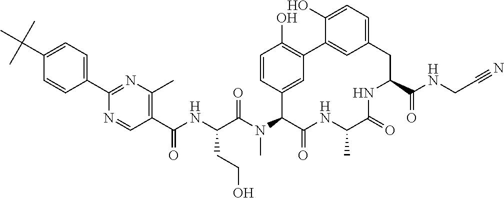 [SMILES: Cc1nc(-c2ccc(C(C)(C)C)cc2)ncc1C(=O)N[C@@H](CCO)C(=O)N(C)[C@@H]1C(=O)N[C@@H](C)C(=O)N[C@H](C(=O)NCC#N)Cc2ccc(O)c(c2)-c2cc1ccc2O] | G8126 | >64 | n.r. | 32 | n.r. | n.r. |
| 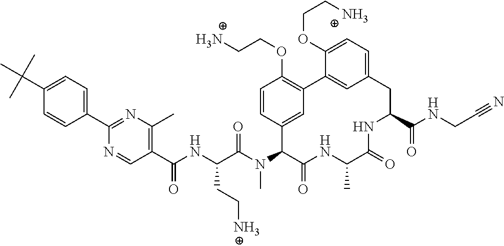 [SMILES: Cc1nc(-c2ccc(C(C)(C)C)cc2)ncc1C(=O)N[C@@H](CC[NH3+])C(=O)N(C)[C@@H]1C(=O)N[C@@H](C)C(=O)N[C@H](C(=O)NCC#N)Cc2ccc(OCC[NH3+])c(c2)-c2cc1ccc2OCC[NH3+]] | G0775 | 2 | n.r. | 0.125 | 0.063 | n.r. |

Development of a fusidic acid derivative with antipseudomonal activity. With an improved understanding of the physicochemical properties that promote intracellular accumulation, as well as the porin-independent mode of uptake of these compounds, we were interested in applying these findings to the development of a novel antipseudomonal. Our discoveries indicate that increasing the hydrogen bond donor ability and the formal charge and/or positive polar surface area of an antibiotic candidate should lead to improved whole-cell antibacterial activity against P. aeruginosa if target binding is not disrupted.

As stated previously, there are many broad-spectrum (Table 7) and Gram-positive-only antibiotics that would have activity against P. aeruginosa if they could achieve higher intracellular accumulation. Fusidic acid (FA), a potent Gram-positive-only antibiotic is one such example. FA is negatively charged and has low hydrogen bond donor surface area, resulting in poor intracellular accumulation and negligible whole-cell antibacterial activity in P. aeruginosa with an MVIC value of 1024 μg/mLh versus PAO1 (Chart 6) and essentially no activity in clinical isolates. Notably, FA has an MIC of 4 μg/mL against the hyperporinated P. aeruginosa A6 efflux deficient strain (PAO1 pore-A6, Chart 6), suggesting that its biological target (elongation factor G, EF-G) is conserved in P. aeruginosa, and that if it could achieve higher intracellular concentrations, it would exhibit whole-cell antibacterial activity.

TABLE 7
Many broad-spectrum antibacterials have limited efficacy against P. aeruginosa
due to poor accumulation, not due to a lack of target homology. In an efflux
pump deficient strain of P. aeruginosa, PAΔ6, antibiotics demonstrate
significantly improved activity compared to the wild-type (WT) strain P. aeruginosa
PAO1, along with increased intracellular accumulation. MICs were performed in
MH or LB broth per Clinical and Laboratory Standards Institute (CLSI) guidelines.
Accumulation units are reported in nmol/10¹² CFUs. The s.e.m is reported
for accumulation values. All experiments were performed in biological
triplicate.

| | WT MIC | PAΔ6 MIC | Fold | WT | PAΔ6 | Fold |
|---|---|---|---|---|---|---|
| Compound | (μg/mL) | (μg/mL) | change | Accum | Accum | change |
| Chloramphenicol | >512 | 4 | >128 | 122 ± 23 | 537 ± 19 | 4.4 |
| Trimethoprim | >512 | 2 | >256 | 136 ± 11 | 356 ± 28 | 2.6 |
| Sulfamethoxazole | >64 | 4 | >16 | 101 ± 26 | 420 ± 15 | 4.2 |
| Tetracycline | 16 | 0.25 | 64 | 452 ± 5 | n.v. | — |

n.v. = not viable.

Chart 6. FA has potent activity against Gram-positive bacteria, but no activity against P. aeruginosa PAO1. However, in the permeabilized and efflux pump knockout (KO) strain of P. aeruginosa, pore-A6, FA has good antibacterial activity, indicating that the target (EF-G) is conserved, and if FA could accumulate, it would be active against wild-type P. aeruginosa. A polyamine version of PA (FA polyamine) lost antibiotic activity but gained accumulation. To combine these features, an amidoxime prodrug moiety was generated with the polyamine linker. The prodrug is cleaved through hydrolysis to release FA inside the cell, leading to an MIC value of 4 g/mL, a 256-fold improvement in activity against wild-type P. aeruginosa.

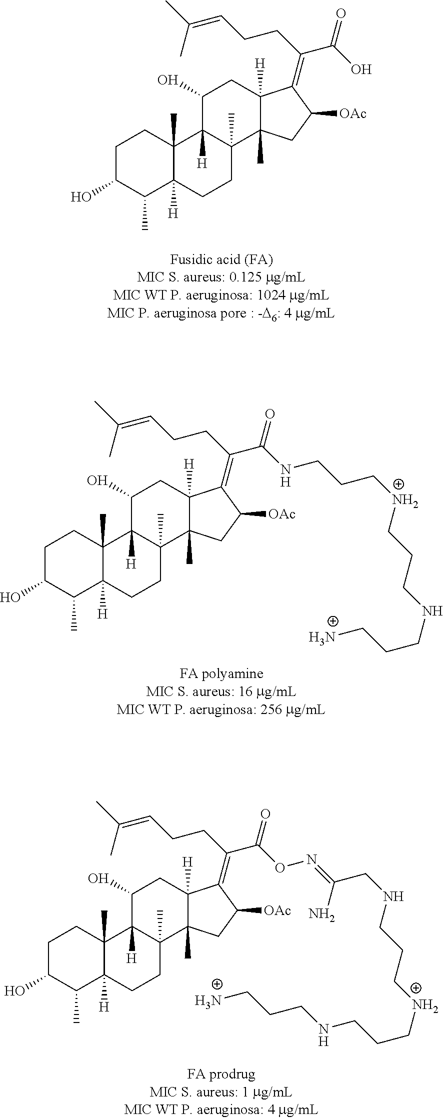

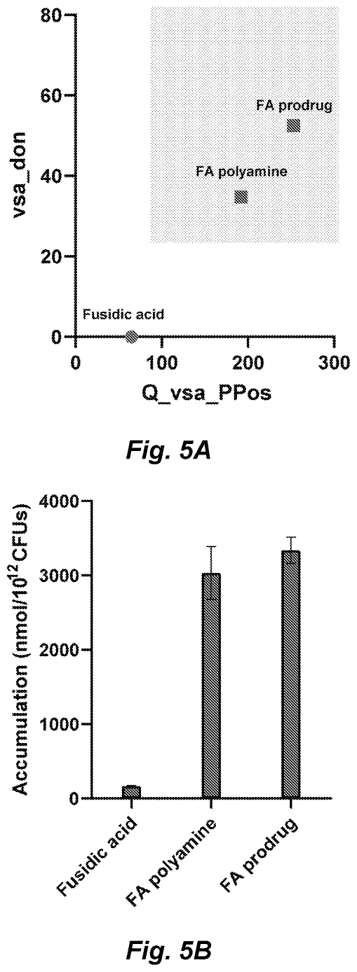

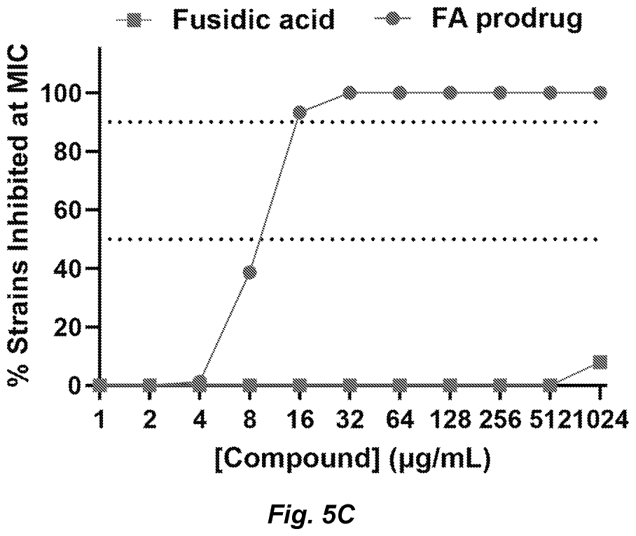

With a vsa_don of 0 and a Q_vsa_PPos of 65.3 (FIG. 5b), FA does not meet the stated guidelines for accumulation in P. aeruginosa and was thus selected as a challenging starting point for conversion to a version active against this pathogen. Efforts towards a Gram-negative active version of FA led first to the design and synthesis of FA polyamine (Chart 6). While FA polyamine no longer has potent antibacterial activity (Chart 6), as the acid is necessary for target engagement, FA polyamine fits the established guidelines for accumulation in P. aeruginosa (FIG. 5b). Encouragingly, this compound shows a 30-fold increase in accumulation relative to FA (FIG. 5c). Inspired by this substantial increase in whole-cell accumulation, we envisioned using a prodrug approach to maintain good permeation into the cell using amine linkers, while also restoring on-target activity against EF-G through the release of free FA inside the cell. As a result, amidoxime prodrug versions containing variable amine linkers were designed (Table 8). The prodrug moiety is cleaved through hydrolysis and results in free FA—In CAMH at 37° C., FA prodrug hydrolyzes to fusidic acid over a period of 48 hours. Data collected in P. aeruginosa PAO1 and E. coli MG1655 demonstrated an increase in activity and accumulation with additional positive charges (Table 8). The version with four ionizable nitrogens (FA prodrug, FIG. 4a) demonstrates the highest accumulation and antibacterial activity (Table 8).

TABLE 8
Introducing a hydrolyzable amidoxime linker onto FA provides a strategy to increase
Gram-negative activity and accumulation. Increasing the number of amines on the linker results in
improved antibacterial activity and accumulation in two Gram-negative species, E. coli MG1655 and
P. aeruginosa PAO1, with the 4-amine linker-containing FA derivative (FA prodrug) demonstrating
the most potent activity. MICs are reported in ug/mL and were performed according to CLSI
guidelines. Accumulation values are reported in nmol/1012 CFUs. All compounds were tested in
biological triplicate.

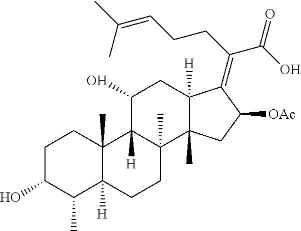

FA

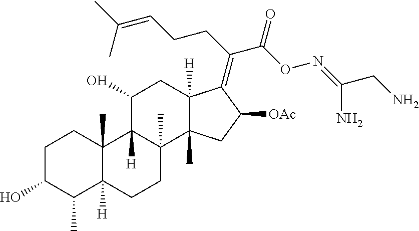

FA prodrug 1

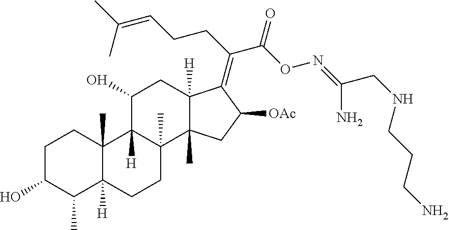

FA prodrug 2

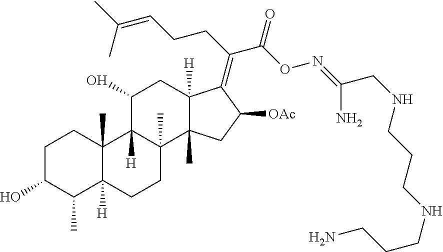

FA prodrug 3

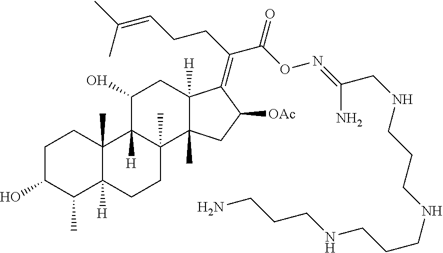

FA prodrug

| | FA | FA prodrug 1 | FA prodrug 2 | FA prodrug 3 | FA prodrug |
|---|---|---|---|---|---|
| E. coli MG1655 MIC | 512 | >256 | 256 | 32 | 4 |
| P. aeruginosa PAO1 MIC | 1024 | >256 | >256 | 128 | 4 |
| EC Accumulation (FA) | 52 ± 5 | 850 ± 61 | 969 ± 71 | 1764 ± 37 | 3337 ± 175 |
| PA Accumulation (FA) | 80 ± 7 | TBD | TBD | TBD | 3396 ± 122 |

FA prodrug meets the vsa_don and Q_vsa_PPos requirements for accumulation in P. aeruginosa (FIG. 4b) and does indeed achieve very high accumulation levels in PAO1 (FIG. 4c; accumulation of FA reported as the prodrug hydrolyzes). Importantly, FA prodrug has an MIC of 4 g/mL against P. aeruginosa PAO1, a 256-fold increase relative to FA (FIG. 4a), and also has activity of 4-16 g/mL against a panel of 75 clinical isolates of P. aeruginosa; FA has essentially no antibacterial activity against this clinical isolate panel (summary in FIG. 5d; all MIC values in Table 11 of Example 1).

FA prodrug hydrolyzes in media too rapidly for isolation of resistance mutants. Instead, resistance mutants to FA were generated in the strain P. aeruginosa PAO1 A6. MIC fold changes of 32-128× were observed for FA in these resistant strains, while FA prodrug exhibited a several fold change. Sequencing offfusA in resistant strains revealed amino acid substitutions xx, which are residues that sit in the FA binding pocket. Further, an FA derivative was synthesized that has the two free alcohols on the core scaffold acetylated, and this compound loses antibacterial activity. Next, a version of this diacylated compound was constructed with the prodrug linker as an additional control for on-target activity (Table 9). This compound loses substantial antibacterial activity relative to FA prodrug, indicating that most of the antibacterial activity of FA prodrug is derived from the inhibition of elongation factor G.

TABLE 9
Acetylating both alcohols of FA disrupts target engagement of FA with EF-G. The
diacetylated version of FA prodrug subsequently loses 4-16× activity against both Gram-positive
and Gram-negative bacterial strains, indicating that inhibition of EF-G is a major contributor to the
observed antibacterial activity of FA prodrug.

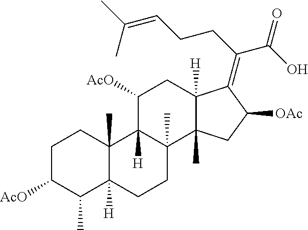

FA diacetate

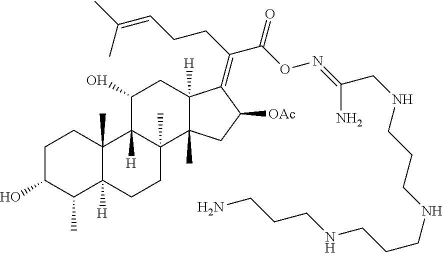

FA prodrug

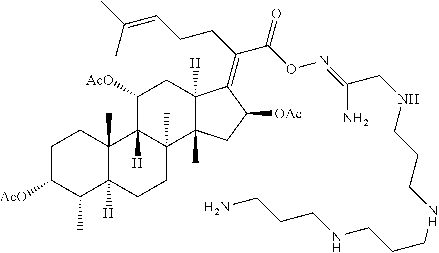

FA prodrug-diacetate

| MIC (μg/mL) | FA | FA diacetate | FA prodrug | FA prodrug-diacetate |
|---|---|---|---|---|
| S. aureus ATCC 29213 | 0.125 | 64 | 1 | 16 |
| E. coli MG1655 | 512 | >512 | 4 | 16 |
| P. aeruginosa PAO1 | 1024 | n.d. | 4 | 32 |

There is also a secondary mechanism of antibacterial action for these compounds, apparent from the residual activity of FA prodrug against resistant strains, the activity of FA polyamine, and the activity of the diacylated prodrug. Based on the polyamine structure, it was hypothesized that the membrane-based interactions may be contributing to this secondary antibacterial activity. A variety of experiments were performed to characterize such interactions. Co-treatment with 5 mM MgCl₂ led to a 32-fold shift in MIC for FA prodrug and gentamicin in P. aeruginosa PAO1, while FA showed no change in antibacterial activity, suggesting that FA prodrug entry into P. aeruginosa occurs via the self-promoted uptake pathway (Table 10).

TABLE 10
Co-treatment with magnesium ions leads to a 32× increase in
MIC for gentamicin and FA prodrug, while FA shows no change. MICs
performed according to CLSI guidelines in biological triplicate.

| | FA | Gentamicin | FA prodrug |
|---|---|---|---|
| P. aeruginosa PAO1 | | | |
| CAMH | 1024 | 0.25 | 4 |
| 5 mM MgCl₂/CAMH | 1024 | 8 | 128 |
| Fold change MIC | 0X | 32X | 32X |

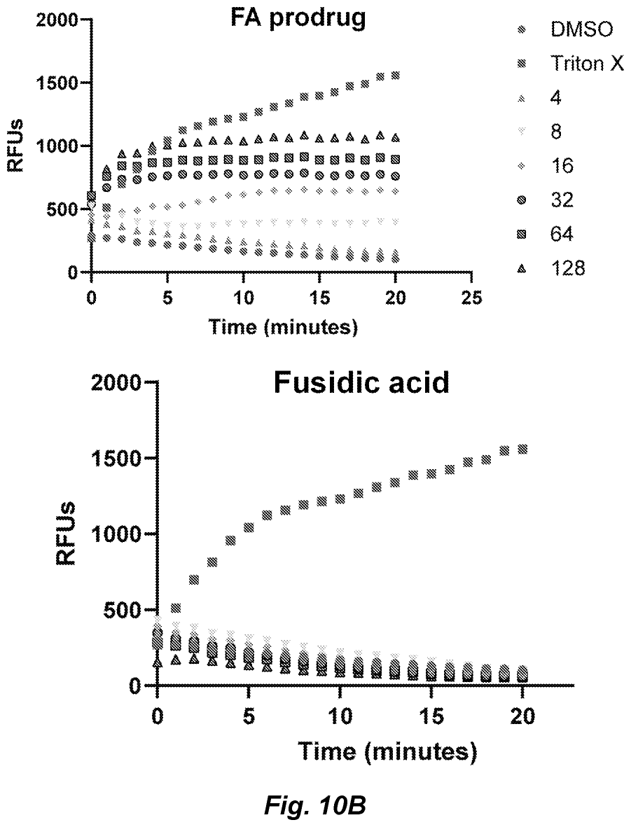

Further, the fluorescent probe N-phenylnapthylamine (NPN) was used to assess outer membrane permeability changes upon treatment with FA, gentamicin, and FA prodrug. NPN is membrane impermeable and is only weakly fluorescent in aqueous solutions. Upon entry to the cell and interacting with phospholipids, significant fluorescence is observed. While treatment of P. aeruginosa PAO1 with FA for ten minutes did not lead to any change in fluorescence, treatment with both gentamicin and FA prodrug led to a dose-dependent increase (FIG. 10b). Finally, using the potentiometric dye DiSC₃ (5), it was demonstrated that treatment with FA prodrug leads to dose-dependent inner membrane depolarization, monitored by an increase in fluorescence, while treatment with FA did not impact membrane polarization. All of this data is consistent with FA prodrug relying on membrane interactions to enter the cell; in addition to engaging EF-G to kill the bacterial cells, the inner membrane depolarization by the FA prodrug is likely also causing some of the observed antibacterial activity (FIG. 10c). This residual membrane depolarizing antibacterial activity can be observed in the FA polyamine (FIG. 4a) and the diacylated compound (Table 9).

### DISCUSSION

Comparison of results from the unbiased accumulation experiments between E. coli and P. aeruginosa highlights the benefit of ionizable nitrogens (with minimal steric encumbrance) and high amphiphilic moment to promote intracellular accumulation in both E. coli and P. aeruginosa. As the eNTRy rules were found to be only 40-50% predictive in P. aeruginosa, chemical traits specific for this species were identified. Vsa_don and positive charge parameters were found to be positively correlated with accumulation in P. aeruginosa, and ~80% of compounds that meet the appropriate cutoffs accumulate. Going forward, these accumulation trends provide a strategy for designing P. aeruginosa-active antibiotics. As evidenced by this study, the unbiased evaluation of non-antibiotic compounds can identify properties that are not apparent in the assessment of antibiotics.

An interesting and surprising aspect of this study is the observed porin-independent uptake of compounds in P. aeruginosa. While many Gram-negative species with general diffusion porins (such as E. coli) have significant data in the literature pointing towards porin-dependent uptake for many compounds, the scope of compound uptake through the substrate-specific channels present in P. aeruginosa has been insufficiently explored. Assessing accumulation herein of non-antibiotics in a strain of P. aeruginosa with 40 putative porins knocked out (A40) demonstrates few significant accumulation differences in the absence of porins. Consistent with our result, a similar trend was observed in assessing MIC values for antibiotics in the wild-type and A40 strain; aside from the carbapenem antibiotics, no antibiotics had an MIC fold change in this study. Collectively, these results suggest that in contrast to E. coli, where the general porin OmpF facilitates uptake for many monoamines, in P. aeruginosa the self-promoted uptake pathway may be the primary mode of uptake for all positively charged compounds. This hypothesis was corroborated by accumulation data with co-treatment of magnesium, leading to a significant reduction in accumulation for most compounds. Multicharged antibiotics, such as the aminoglycosides and colistin, have been suggested to utilize this pathway for uptake into bacteria, but the utility of this pathway for monoamine uptake had not yet been disclosed. This demonstration of non-porin mediated uptake suggests the critical importance of permeation through the outer membrane, specifically in P. aeruginosa, and is significant for the design and optimization of high-accumulating antipseudomonals.

The development of a prodrug moiety that can facilitate self-promoted uptake into P. aeruginosa for fusidic acid may prove generalizable for other antibiotics where antibacterial activity is limited due to poor intracellular accumulation. While antibacterial prodrug approaches have been described that rely on bacterial enzymes for prodrug processing and activation, resistance via mutation of the activating enzyme can confound such strategies. The reliance herein on a hydrolyzable linkage obviates this potential resistance mechanism, with the prodrug facilitating uptake before its hydrolytic removal. Of note, hydrolytic prodrug strategies can be effective clinically as demonstrated by the anticancer drug temozolomide. Numerous other Gram-positive-only antibiotics or preclinical candidates possess a carboxylic acid that could be used with this same prodrug approach, and adaptations to other functional groups can be envisioned.

Certain historical data and trends from the literature begin to make more sense in light of the results presented herein. For example, the impenetrability of P. aeruginosa, even relative to other Gram-negative bacteria, has been noted by others and is powerfully shown (in comparison to E. coli) in FIG. 8. As certain small molecules can enter E. coli (but not P. aeruginosa) through porins, the general impenetrability of P. aeruginosa appears to be linked to the squelching of any porin-dependent uptake pathways. The inability to exploit porin-mediated uptake in P. aeruginosa has stymied antibiotic development for this pathogen, but data presented herein suggests that purposeful design of antibiotics to leverage porin-independent mechanisms can be fruitful in the generation of novel antibiotics for this dangerous pathogen.

### General Synthetic Methods

The invention also relates to methods of making the compounds and compositions of the invention. The compounds and compositions can be prepared by any of the applicable techniques of organic synthesis, for example, the techniques described herein. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as standard organic reference texts such as March's Advanced Organic Chemistry: Reactions, Mechanisms, and Structure, 5^{th} Ed. by M. B. Smith and J. March (John Wiley & Sons, New York, 2001), Comprehensive Organic Synthesis; Selectivity, Strategy & Efficiency in Modern Organic Chemistry, in 9 Volumes, Barry M. Trost, Ed.-in-Chief (Pergamon Press, New York, 1993 printing)); Advanced Organic Chemistry, Part B: Reactions and Synthesis, Second Edition, Cary and Sundberg (1983); Protecting Groups in Organic Synthesis, Second Edition, Greene, T. W., and Wutz, P. G. M., John Wiley & Sons, New York; and Comprehensive Organic Transformations, Larock, R. C., Second Edition, John Wiley & Sons, New York (1999).

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically, the temperatures will be −100° C. to 200° C., solvents will be aprotic or protic depending on the conditions required, and reaction times will be 1 minute to 10 days. Work-up typically consists of quenching any unreacted reagents followed by partition between a water/organic layer system (extraction) and separation of the layer containing the product.

Oxidation and reduction reactions are typically carried out at temperatures near room temperature (about 20° C.), although for metal hydride reductions frequently the temperature is reduced to 0° C. to −100° C. Heating can also be used when appropriate. Solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions). Standard synthetic techniques such as azeotropic removal of reaction by-products and use of anhydrous reaction conditions (e.g., inert gas environments) are common in the art and will be applied when applicable. Amidoxime esters are formed using methods familiar to a person of ordinary skill in the art, for example by combining an oxime and an acid or derivative thereof.

Protecting Groups. The term “protecting group” refers to any group which, when bound to a hydroxy or other heteroatom prevents undesired reactions from occurring at this group and which can be removed by conventional chemical or enzymatic steps to reestablish the hydroxyl group. The particular removable protecting group employed is not always critical and preferred removable hydroxyl blocking groups include conventional substituents such as, for example, allyl, benzyl, acetyl, chloroacetyl, thiobenzyl, benzylidene, phenacyl, methyl methoxy, silyl ethers (e.g., trimethylsilyl (TMS), t-butyl-diphenylsilyl (TBDPS), or t-butyldimethylsilyl (TBS)) and any other group that can be introduced chemically onto a hydroxyl functionality and later selectively removed either by chemical or enzymatic methods in mild conditions compatible with the nature of the product.

Suitable hydroxyl protecting groups are known to those skilled in the art and disclosed in more detail in T. W. Greene, Protecting Groups In Organic Synthesis; Wiley: New York, 1981 (“Greene”) and the references cited therein, and Kocienski, Philip J.; Protecting Groups (Georg Thieme Verlag Stuttgart, New York, 1994), both of which are incorporated herein by reference.

Protecting groups are available, commonly known and used, and are optionally used to prevent side reactions with the protected group during synthetic procedures, i.e. routes or methods to prepare the compounds by the methods of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin. Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user. Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-bacterial agents. Preferably, compounds of the invention are less prone to resistance by gram-negative bacteria.

The invention provides therapeutic methods of treating an infection in a mammal, which involve administering to a mammal having cancer an effective amount of a compound or composition described herein. A mammal includes a primate, human, rodent, canine, feline, bovine, ovine, equine, swine, caprine, bovine and the like.

The ability of a compound of the technology to treat infections may be determined by using assays well known to the art. For example, the design of treatment protocols, toxicity evaluation, data analysis, quantification of cell kill are known.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Experimental Methods

Bacterial Strains. P. aeruginosa (AR, Cubist, PAO1, PAO1 A6, PA14, PA14 A40) and E. coli MG1655.

Antimicrobial Susceptibility Tests. Susceptibility testing was performed in biological triplicate, using the microdilution broth method as outlined by the Clinical and Laboratory Standards Institute. Bacteria were cultured with cation-adjusted Meuller-Hinton broth (Sigma-Aldrich; catalogue number: 90922) or Luria-Bertani (LB) broth (Lennox) in round-bottom 96-well plates (Corning; catalogue number: 3788).

Accumulation Assay. The accumulation assay was performed in triplicate in batches of ten samples, with each batch containing tetracycline or ciprofloxacin as a positive control. P. aeruginosa PAO1, PAO1 A6, PA14, PA14 A40, and PA1280, and E. coli MG1655 were used in these experiments. For each replicate, 2.5 mL (E. coli) or 5 mL (P. aeruginosa) of an overnight culture was diluted into 250 mL of fresh Luria-Bertani broth (Lennox) or Tryptic Soy Broth (TSB) and grown at 37° C. with shaking to an optical density (OD600) of 0.55-0.60. The bacteria were pelleted at 3220 r.c.f. for 10 min at 4° C., and the supernatant was discarded. The pellets were resuspended in 40 mL of phosphate buffered saline (PBS) and pelleted as before, and the supernatant was discarded. The pellets were resuspended in 8.8 mL of fresh PBS and aliquoted into ten, 1.5 mL Eppendorf tubes (875 μL each). The number of colony-forming units (CFUs) was determined by a calibration curve. The samples were equilibrated at 37° C. with shaking for 5 min; compound was added (final concentration=50 μM), and then the samples were incubated at 37° C. with shaking for either 10 min. These time points were short enough to minimize metabolic and growth changes (no changes in OD₆₀₀ or CFU count observed). After incubation, 800 μL of the cultures was carefully layered on 700 μL of silicone oil (9:1 AR20/Sigma High Temperature, cooled to −78° C.). Bacteria were pelleted through the oil by centrifuging at 13 000 r.c.f. for 2 min at room temperature (with the supernatant remaining above the oil); the supernatant and oil were then removed by pipetting. To lyse the samples, each pellet was dissolved in 200 L of water, and then, they were subjected to three (E. coli) or four (P. aeruginosa) freeze-thaw cycles of 3 min in liquid nitrogen followed by 3 min in a water bath at 65° C. P. aeruginosa samples were then treated with 50 M DNase and RNase and incubated at 37° C. for 15 minutes. The lysates were pelleted at 13 000 r.c.f. for 2 min at room temperature, and the supernatant was collected (180 L). The debris was resuspended in 100 μL of methanol and pelleted as before. The supernatants were removed and combined with the previous supernatants collected. Finally, the remaining debris was removed by centrifuging at 20 000 r.c.f. for 10 min at room temperature. Supernatants were analyzed by LC-MS/MS.

Samples were analyzed with the 5500 QTRAP LC/MS/MS system (AB Sciex) with a 1200 series HPLC system (Agilent Technologies) including a degasser, an autosampler, and a binary pump. Liquid chromatography separation was performed on an Agilent SB-Aq column (4.6×50 mm, 5 μm) (Agilent Technologies) with mobile phase A (0.1% formic acid in water) and mobile phase B (0.1% formic acid in acetonitrile). The flow rate was 0.3 mL/min. The linear gradient was as follows: 0-3 min, 100% mobile phase A; 10-15 min, 2% mobile phase A; 15.5-21 min, 100% mobile phase A. The autosampler was set at 5° C. The injection volume was 15 μL. Mass spectra were acquired with both positive electrospray ionization at the ion spray voltage of 5500 V and negative electrospray ionization at the ion spray voltage of −4500 V. The source temperature was 450° C. The curtain gas, ion source gas 1, and ion source gas 2 were 33, 50, and 65, respectively. Multiple reaction monitoring was used to quantify the metabolites. Power analysis was not used to determine the number of replicates. Error bars represent the standard error of the mean of three biological replicates. The statistical significance of the accumulation was determined using a two sample Welch's t test (one-tailed test, assuming unequal variance) relative to the negative controls. All compounds evaluated in the biological assays were >95% pure.

MBN assay. Assays measuring permeabilization by polymyxin B nonapeptide (PMBN) were performed as above, with the addition of 8 μg/mL PMBN immediately before the compound of interest was added.

Accumulation assay with magnesium. Assays measuring impact of magnesium treatment were performed as above, with the addition of 1 mM MgCl₂ immediately before the compound of interest was added.

Selection ofresistant mutants. Resistant mutants were selected using the large inoculum method. Briefly, P. aeruginosa Δ6 (1.8×10⁹ c.f.u.) was plated on 100 mm plates of Luria Bertani agar containing 64, 32 or 16 g ml-1 fusidic acid with 10 g/mL gentamicin. Colonies were visible after incubating at 37° C. for 72 h. Resistant colonies were confirmed by streaking on selective media with the same concentration of fusidic acid.

Calculation of Physiochemical Properties. Data sets of chemical structures were created and managed using Maestro.46 Initial structure preparation and 3D minimization were performed with LigPrep47 using OPLS_2005 force fields. The generation of ensembles of conformations was performed using Macromodel,48 using the OPLS3 force field, with solvation (water). The search method used was the mixed torsional/Low mode sampling, and the energy cutoff was set to 10 kcal/mol. All other options were left as defaults. Physiochemical descriptors (both 2D- and 3D-based) were calculated using MOE 2015.1049 for each conformation. Descriptors were averaged (unweighted mean) across all conformations for each molecule.

Statistical analyses. GraphPad Prism 8.2.1.441 was used for data analysis and figure generation. Data are shown as means s.e.m. Statistical significance was determined by one-way ANOVA (with Tukey's multiple comparisons test) for two groups at a single time point, two-way ANOVA (with Sidak's multiple comparison's test) for two groups at multiple time points, or two-way ANOVA (with Tukey's multiple comparisons test) for three or more groups at multiple time points. P<0.05 was considered statistically significant. In this study, no statistical methods were used to predetermine the sample size. The experiments were not randomized, and the investigators were not blinded to allocation during the experiments and outcome assessments.

TABLE 11
Fusidic acid and FA prodrug tested against 75 clinical isolates
of P. aeruginosa. MICs were performed according to CLSI guidelines.
All compounds were tested in biological triplicate.

MIC (μg/mL)

| Bacterial strain | Fusidic acid | FA prodrug |
|---|---|---|
| Reference strains | | |
| PAO1 | 1024 | 4 |
| PA14 | >1024 | 8 |
| AR Bank Isolate # | | |
| 0054 | >1024 | 16 |
| 0064 | >1024 | 16 |
| 0090 | >1024 | 16 |
| 0092 | >1024 | 16 |
| 0095 | >1024 | 16 |
| 0100 | >1024 | 16 |
| 0103 | >1024 | 16 |
| 0110 | >1024 | 16 |
| 0229 | >1024 | 16 |
| 0230 | >1024 | 16 |
| 0231 | >1024 | 8 |
| 0232 | >1024 | 16 |
| 0233 | >1024 | 16 |
| 0234 | 1024 | 8 |
| 0235 | >1024 | 8 |
| 0236 | >1024 | 16 |
| 0237 | >1024 | 16 |
| 0239 | >1024 | 8 |
| 0240 | >1024 | 16 |
| 0241 | >1024 | 16 |
| 0242 | >1024 | 8 |
| 0243 | >1024 | 16 |
| 0244 | >1024 | 16 |
| 0245 | >1024 | 16 |
| 0246 | 1024 | 8 |
| 0248 | >1024 | 8 |
| 0250 | >1024 | 16 |
| 0252 | >1024 | 16 |
| 0254 | >1024 | 32 |
| 0265 | >1024 | 16 |
| 0763 | >1024 | 16 |
| 0767 | >1024 | 16 |
| 0769 | >1024 | 16 |
| 0772 | >1024 | 8 |
| Cubist Strain ID # (n = 22) | | |
| 996 | >1024 | 8 |
| 997 | >1024 | 8 |
| 998 | 1024 | 8 |
| 999 | >1024 | 8 |
| 1000 | >1024 | 16 |
| 1001 | >1024 | 16 |
| 1174 | >1024 | 16 |
| 1175 | >1024 | 16 |
| 1176 | >1024 | 16 |
| 1280 | >1024 | 16 |
| 1282 | >1024 | 16 |
| 1283 | >1024 | 16 |
| 1284 | >1024 | 16 |
| 1586 | >1024 | 16 |
| 1590 | >1024 | 32 |
| 1591 | >1024 | 32 |
| 1592 | >1024 | 32 |
| 1905 | >1024 | 16 |
| 1906 | >1024 | 16 |
| 2242 | >1024 | 8 |
| 2243 | >1024 | 16 |
| 2244 | >1024 | 8 |
| Carle Isolate ID # | | |
| Cl-02 | 1024 | 8 |
| Cl-03 | 1024 | 8 |
| Cl-04 | >1024 | 16 |
| Cl-05 | >1024 | 8 |
| Cl-06 | >1024 | 8 |
| Cl-07 | >1024 | 8 |
| Cl-08 | >1024 | 8 |
| Cl-09 | >1024 | 8 |
| Cl-10 | >1024 | 16 |
| Cl-11 | >1024 | 8 |
| Cl-12 | >1024 | 8 |
| Cl-13 | >1024 | 16 |
| Cl-14 | >1024 | 8 |
| Cl-15 | >1024 | 8 |
| Cl-19 | >1024 | 8 |
| Other strains (ref.) | | |
| 1113 | >1024 | 16 |
| 1727 | >1024 | 8 |

### Example 2. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/ml |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 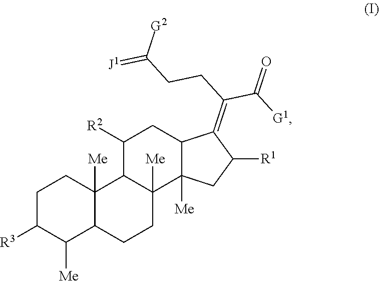
or a salt thereof,
wherein
G¹ is —O—N═CR⁴R⁵, NR⁶R⁷, or OR′;
G² is H, halo, OH, —(C₁-C₆)alkyl, —O(C₁-C₆)alkyl, or NR^{a}R^{b};
J¹ is CR^{c}R^{d}, O, or absent;
R^{a} and R^{b} are each independently H, —(C₁-C₆)alkyl, or —(C₃-C₆)cycloalkyl;
R^{c} and R^{d} are each independently —(C₁-C₆)alkyl, —(C₃-C₆)cycloalkyl, H, halo, aryl, or —(C₀-C₅)R^{e} wherein R^{c} is OH, —O(C₁-C₆)alkyl, or —C(═O)O(C₁-C₆)alkyl; or
R^{c} and R^{d} taken together form a cycloalkyl or heterocycloalkyl;
R¹ is —OC(═O)(C₁-C₆)alkyl, H, methyl, ethyl, hydroxy, methoxy, ethoxy, or amino;
R² and R³ are each independently hydroxy, H, methyl, ethyl, methoxy, ethoxy, amino, or —OC(═O)(C₁-C₆)alkyl;
R⁴ is —CH₂R⁹, aryl, heteroaryl, or alkyl, wherein R⁹ is —(NH(CH₂)₃)₃NH₂ or a different N-polyaminoalkyl, N-monoaminoalkyl, N-heterocycloalkyl, amino, azido, halo, aryl, or heteroaryl;
R⁵ is NR^{a}R^{b}, —(C₁-C₆)alkyl, or H; or
R⁴ and R⁵ taken together from a heterocycloalkyl;
R⁶ is alkylpolyamine or alkylamine;
R⁷ is H or —(C₁-C₆)alkyl; and
R⁸ is alkylenecarbamate or alkylenecarbonate.

2. The compound of claim 1 wherein the compound is represented by Formula II: 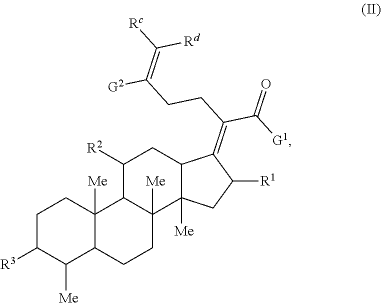
or a salt thereof.

3. The compound of claim 1 wherein G¹ is —O—N═CR⁴R⁵ or G¹ is R⁹.

4. The compound of claim 1 wherein the compound is represented by Formula III: 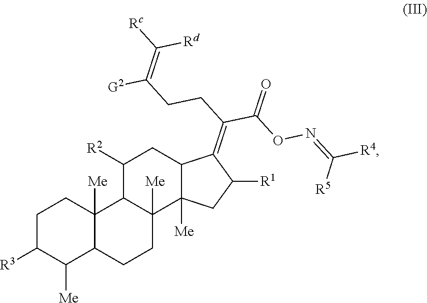
or a salt thereof.

5. The compound of claim 1 wherein R⁴ is —CH₂R⁹.

6. The compound of claim 1 wherein the compound is represented by Formula IV: 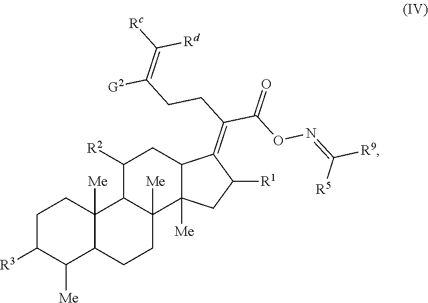
or a salt thereof.

7. The compound of claim 1 wherein J¹ is CR^{c}R^{d}.

8. The compound of claim 7 wherein R^{c} and R^{d} are each independently —(C₁-C₆)alkyl.

9. The compound of claim 1 wherein G² is H.

10. The compound of claim 1 wherein R⁵ is NR^{a}R^{b}, or wherein R⁹ is —(NR^{a}(C₂-C₆)NR^{b})m wherein m is an integer from 1 to 6.

11. The compound of claim 1 wherein R⁹ is NH₂, NHCH₃, —NHCH₂CH₂NH₂, —NHCH₂CH₂CH₂NH₂, —NHCH₂CH₂CH₂CH₂NH₂, —(NHCH₂CH₂)₂NH₂, —(NHCH₂CH₂CH₂)₂NH₂, —(NHCH₂CH₂CH₂CH₂)₂NH₂, —(NHCH₂CH₂)₃NH₂, —(NHCH₂CH₂CH₂)₃NH₂, —(NHCH₂CH₂CH₂CH₂)₃NH₂, —(NHCH₂CH₂)₄NH₂, —(NHCH₂CH₂CH₂)₄NH₂, —(NHCH₂CH₂CH₂CH₂)₄NH₂, —N(CH₂CH₂)₂NH, or NHCH₂Ph wherein Ph is optionally substituted with amino or halo.

12. The compound of claim 1 wherein the compound is: 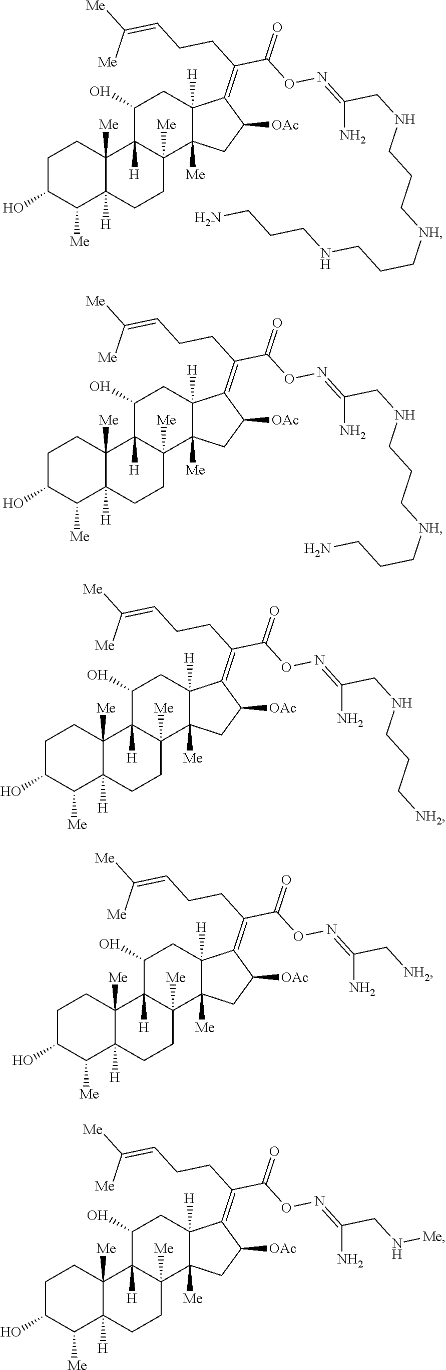 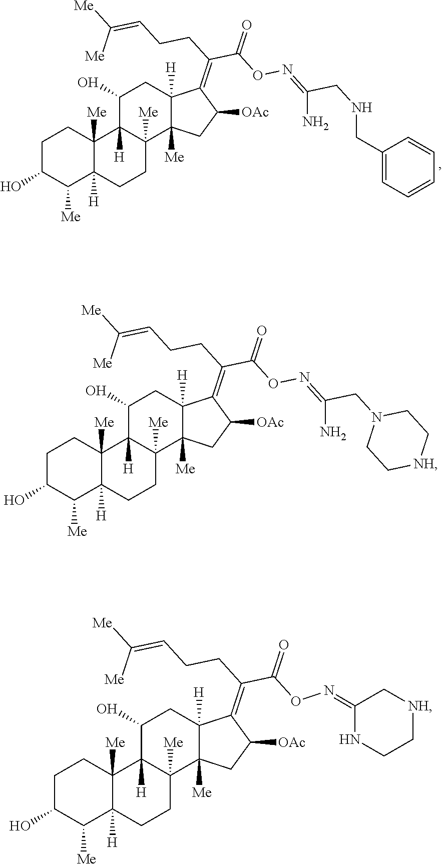
or a salt thereof, or
wherein the compound is: 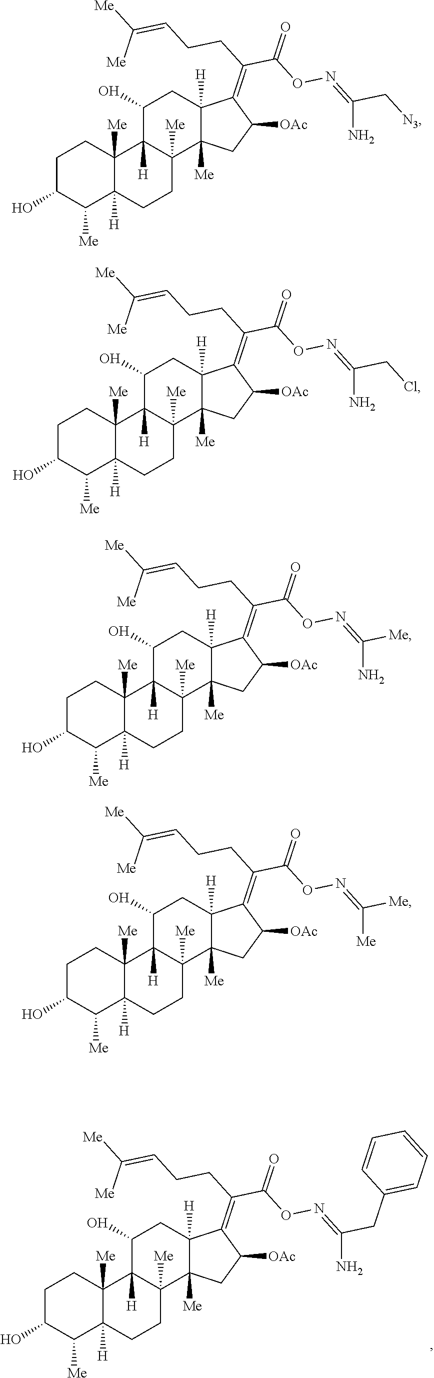 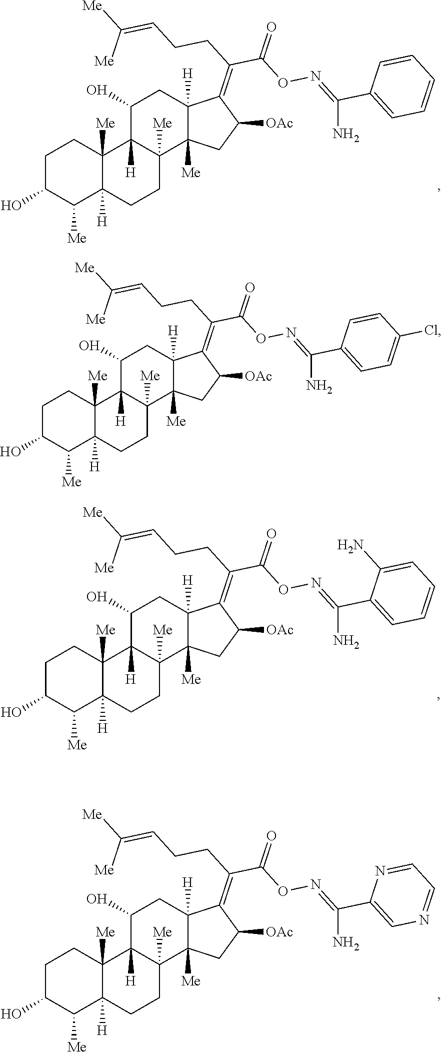
or a salt thereof, or
wherein the compound is: 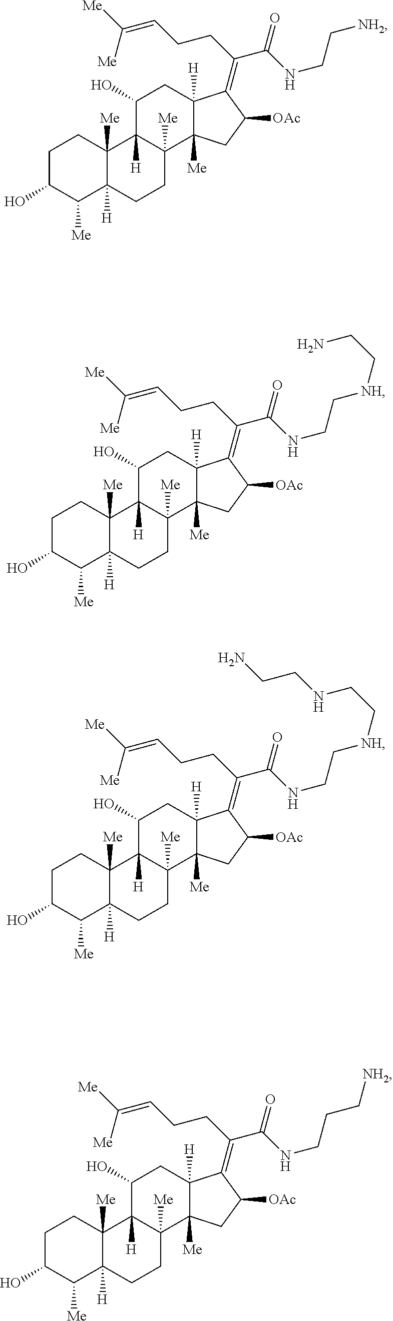 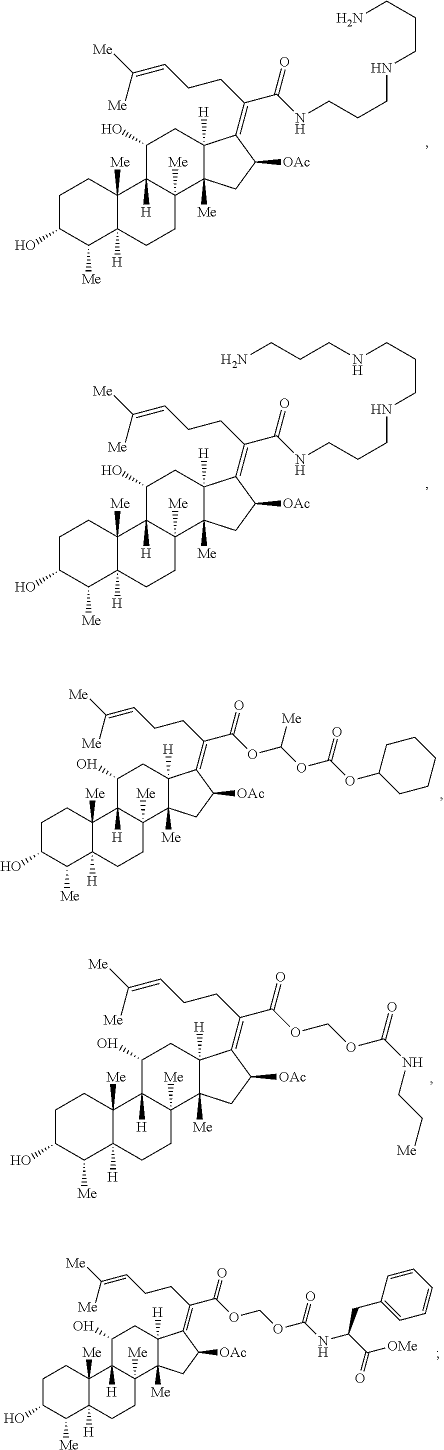
or a salt thereof.

13. A method of antimicrobial treatment comprising administering to a subject in need thereof a therapeutically effective amount of a compound of claim 1 or a pharmaceutically acceptable salt thereof, thereby killing or inhibiting the growth of at least a portion of a plurality of bacteria in the subject, wherein optionally the bacteria is Gram-negative bacteria.

14. The method of claim 13 wherein the bacteria is Pseudomonas aeruginosa, Enterobacteriaceae, Acinetobacter baumannii, or a combination thereof; or
wherein the bacteria is Acinetobacter, anthrax-causing bacteria, Bacilli, Bordetella, Borrelia, botulism-causing bacteria, Brucella, Burkholderia, Campylobacter, Chlanydica, cholera-causing bacteria, Clostichium, Conococcus, Corynebacteriun, diptheria-causing bacteria, Enterobacter, Enterococcus, Erwinia, Escherichia, Francisella, Haemophilus, Heilobacter; Klebsiella, Legionella, Leptospira, leptospirosis-causing bacteria, Listeria, Lyme's disease-causing bacteria, meningococcus, Mycobacterium, Mycoplasma, Neisseria, Pasteurella, Pelobacter, plague-causing bacteria, Pneurnonococcus, Proteus, Pseudononas, Rickettsia, Salmonella, Serratia, Shigella, Staphylococcus, Streplococcus, tetanus, Treponenma, Vibrio, Yersinia, Xanthomnonas, or a combination thereof

15. The method of claim 13 wherein the compound is:
(3R, 4S,5S,8S,9S,10S,11R,13R,14S,16S,Z)-17-((Z)-1,14-diamino-22-methyl-17-oxo-16-oxa-4,8,12,15-tetraazatricosa-14,21-dien-18-ylidene)-3,11-dihydroxy-4,8,10,14-tetramethylhexadecahydro-1H-cyclopenta[a]phenanthren-16-yl acetate.
